(19) United States
(12) Patent Application Publication
(10) Pub. No.: US 2025/0040852 A1
(43) Pub. Date: Feb. 6, 2025

(54) SYSTEMS AND METHODS FOR COCHLEAR TRAUMA MANAGEMENT DURING AN ELECTRODE LEAD INSERTION PROCEDURE

(71) Applicant: Advanced Bionics LLC, Valencia, CA (US)

(72) Inventors: Sridhar Kalluri, Pasadena, CA (US); Saba Farbodkia, Kitchener (CA); Kanthaiah Koka, Valencia, CA (US)

(21) Appl. No.: 18/718,210

(22) PCT Filed: Dec. 30, 2021
(86) PCT No.: PCT/US2021/065611

§ 371 Date: Jun. 10, 2024

(51) Int. Cl. A61B 5/279; A61B 5/00; A61N 1/04

(52) U.S. Cl. CPC A61B 5/279; A61B 5/6846; A61N 1/04

(57) ABSTRACT

An illustrative cochlear trauma management system determines, intraoperatively during an insertion procedure to introduce an electrode lead of a cochlear implant system into a cochlea of a recipient, an electrical potential evoked in response to acoustic stimulation applied to the recipient as part of a diagnostic test. The system estimates a depth of the electrode lead within the cochlea for the diagnostic test and accesses recipient attribute data that represents a hearing attribute of the recipient. The recipient attribute data is generated based on preoperative analysis of the recipient. Based on the electrical potential and the recipient attribute data, the system intraoperatively determines a likelihood that the electrode lead is inflicting trauma on the cochlea at the estimated depth. The system then performs an action based on the likelihood that the electrode lead is inflicting the trauma on the cochlea. Corresponding systems and methods are also disclosed.

### BACKGROUND INFORMATION

People who have little or no natural hearing may benefit from a cochlear implant system that stimulates auditory nerves in ways that natural hearing mechanisms fail to stimulate for various reasons. For example, an electrode lead may be inserted into a cochlea of a recipient and stimulation current may be applied by electrodes on the lead as directed by a cochlear implant that is surgically implanted within the recipient.

The insertion of an electrode lead into the cochlea of the recipient is performed by way of a delicate surgical procedure that can cause trauma to the cochlea of the patient. As a result, it may be desirable for surgeons and/or other professionals assisting with introducing the electrode lead into the cochlea to monitor the cochlea during the insertion procedure to detect trauma to the cochlea in real-time as the insertion procedure takes place. For example, electrical potentials generated by the cochlea as evoked responses to diagnostic stimuli may be recorded to provide information to the surgical team about the status of the insertion procedure (status that would be difficult to ascertain other ways due to limited visibility, etc.). By successfully monitoring and managing (e.g., avoiding, mitigating, reducing, etc.) cochlear trauma during surgery, the integrity and structure of the cochlea may be preserved, along with residual hearing that the recipient may possess.

Certain approaches to cochlear trauma management have focused on evoked responses to auditory stimuli by interpreting sudden or significant drops in evoked potentials to be indicative of imminent trauma to the cochlea. Unfortunately, such drops of electrical potential, even if properly and timely detected, may not necessarily indicate cochlear trauma. Accordingly, existing cochlear trauma management systems may be prone to false positive feedback, commonly reporting trauma when no trauma actually exists. Though such reporting may still be of value in helping surgical teams perform careful and effective insertion procedures, an abundance of false positive reports can confuse and annoy surgical team members and, ultimately, even reduce their trust in the system. Eventually, the trauma-related information provided by such systems may come to have less influence on the team's decision-making during the procedure, and the effectiveness of the cochlear trauma management may therefore be reduced.

### BRIEF DESCRIPTION OF THE DRAWINGS

The accompanying drawings illustrate various embodiments and are a part of the specification. The illustrated embodiments are merely examples and do not limit the scope of the disclosure. Throughout the drawings, identical or similar reference numbers designate identical or similar elements.

FIG. 1 shows an illustrative cochlear trauma management system configured to perform cochlear trauma management during an electrode lead insertion procedure.

FIG. 2 shows an illustrative method for cochlear trauma management during an electrode lead insertion procedure.

FIG. 3 shows a configuration in which an illustrative cochlear implant system implements and/or interoperates with an illustrative cochlear trauma management system to perform cochlear trauma management during an electrode lead insertion procedure.

FIGS. 4A-4B show different views of a cochlea of a cochlear implant recipient during an illustrative electrode lead insertion procedure.

FIG. 5 shows illustrative actions that a cochlear trauma management system may perform at different surgery phases before, during, and after an electrode lead insertion procedure.

FIG. 6 shows illustrative aspects of recipient attribute data that may represent hearing attributes of a particular recipient and may be determined, stored, and accessed to facilitate cochlear trauma management during an electrode lead insertion procedure.

FIG. 7 shows illustrative aspects of various ways a depth of the electrode lead may be estimated during the electrode lead insertion procedure.

FIG. 8 shows illustrative aspects of how diagnostic tests may be performed during an electrode lead insertion procedure to evoke electrical potentials that can be used for cochlear trauma management.

FIGS. 9-11 show various illustrative ways that measured electrical potentials and recipient attribute data may be used to intraoperatively determine a likelihood that an electrode lead is inflicting trauma on a cochlea at a particular depth within the cochlea.

FIG. 12 shows illustrative aspects of how feedback provided by a cochlear trauma management system may be used to ensure that an insertion procedure is effective and successful.

FIG. 13 shows an illustrative computing system that may implement cochlear trauma management systems and/or other computing systems and devices described herein.

### DETAILED DESCRIPTION

Systems and methods for cochlear trauma management during an electrode lead insertion procedure are described herein. For example, disclosed systems and methods may manage cochlear trauma, during an insertion procedure whereby an electrode lead is introduced into a cochlea of a cochlear implant recipient, by detecting, estimating, and/or otherwise monitoring for cochlear trauma and, when trauma is determined to be present, performing one or more actions to disclose, mitigate, reduce, and/or otherwise address the trauma. While it is worthwhile for many reasons to detect whether cochlear trauma has occurred even after an insertion procedure is complete, it is especially helpful for cochlear trauma management to be performed instantaneously, in real time, as an insertion procedure is ongoing. Such intraoperative, real-time cochlear trauma management may enable evasive action to be taken when trauma is likely to be inflicted. For instance, cochlear trauma management systems described herein may intraoperatively detect cochlear trauma, and, in response to such detections, may make people and/or robotic assistance systems aware of the imminent trauma so that immediate mitigation can be performed (e.g., by ceasing or slowing down insertion of the lead, by partially or fully withdrawing the lead before continuing, etc.), or so that the situation can otherwise be dealt with as may be appropriate under the circumstances.

As described above, conventional cochlear trauma management systems are often prone to false positive feedback that reduces their effectiveness by, among other things, limiting the amount of confidence that is placed in their overactive predictions. One cause for this tendency to falsely predict cochlear trauma is that trauma is predicted based on circumstances that may be present for a variety of reasons, only one of which is imminent cochlear trauma. For example, a conventional cochlear trauma management system may monitor an insertion procedure using electrocochleography (ECochG) or other diagnostic tests configured to apply acoustic stimulation at a particular frequency and to detect evoked potentials emitted within the cochlea in response to this stimulation. Such systems may estimate evoked potential levels that can be expected given a particular depth of the electrode lead and given the frequency of the acoustic stimulation, and may warn of cochlear trauma when this estimate falls far afield of the potentials actually being measured (e.g., indicating that trauma is likely when one or more ECochG tests records electrical potentials far below what is expected to be measured at a certain depth).

Unfortunately, cochlear trauma is only one possible cause for a diagnostic test result to drop below an expected value. Other possible causes for these complex signals to diverge or be misinterpreted relate to factors that are unknown to, and thus not accounted for by, conventional cochlear trauma management systems. For example, one unaccounted-for factor that may affect ECochG or other such diagnostics may be an idiosyncratic distribution of signal generators (e.g., hair cells within the cochlea, etc.) from which electrical potentials originate in response to the acoustic stimulation of the test. Another illustrative factor influencing diagnostic results (but unaccounted for by conventional systems) may be the etiology of hearing loss of the specific recipient.

Accordingly, systems and methods described herein relate to how cochlear trauma management may be performed effectively during an electrode lead insertion procedure by accounting for: 1) an electrical potential recorded as part of a diagnostic test involving acoustic or other stimuli (e.g., an ECochG test or the like); 2) an intraoperatively-estimated electrode lead depth corresponding with the diagnostic test being performed; and 3) any of various types of preoperatively-determined recipient attribute data indicative of how one or more of the factors described above are to be taken in account for the particular recipient and at the particular estimated depth. In this way, systems and methods described herein may effectively distinguish markers in the ECochG signal (or other diagnostic test results) that indicate trauma from markers that reflect atraumatic, idiosyncratic ECochG signal variations. More specifically, systems described herein may intraoperatively manage cochlear trauma based on not only electrical potentials and estimated depths, but also based on a computational auditory model configured to account for other effects on the diagnostic test results (e.g., the ECochG signal) that are not due to trauma. For example, the model may allow the system to account for effects of a recording electrode changing its location relative to the ECochG signal generators as it is inserted by incorporating an estimate of electrode position within the cochlea. Moreover, the model may enable the system to account for idiosyncratic characteristics of the recipient by, for instance, incorporating preoperative audiogram data for the recipient and any measure characterizing the mechanism (e.g., etiology) of cochlear hearing impairment. The auditory model may incorporate the electrode position, preoperative audiogram, hearing loss etiology, and/or any other suitable factors into the recipient attribute data that is used in determining the likelihood of imminent cochlear trauma.

Leveraging all of this information in the ways described herein, disclosed systems and methods for cochlear trauma management may effectively detect and handle cochlear trauma events without misinterpreting changes in electrical potential that may not be due to trauma (e.g., when a recording electrode crosses the characteristic frequency location of a stimulus tone, when a recording electrode passes through an isolated region within the cochlea where there are more or fewer surviving hair cells than in surrounding regions, etc.). By determining and acting on cochlear trauma more accurately in these ways, intraoperative cochlear trauma management systems and methods described herein may be more widely accepted, trusted, and relied on during crucial insertion procedures, thereby leading to an increased likelihood of positive surgical outcomes for both the recipients receiving the procedures and the medical practitioners performing the procedures.

Various specific embodiments will now be described in detail with reference to the figures. It will be understood that the specific embodiments described below are provided as non-limiting examples of how various novel and inventive principles may be applied in various situations. Additionally, it will be understood that other examples not explicitly described herein may also be captured by the scope of the claims set forth below. Systems and methods described herein for cochlear trauma management during an electrode lead insertion procedure may provide any of the benefits mentioned above, as well as various additional and/or alternative benefits that will be described and/or made apparent below.

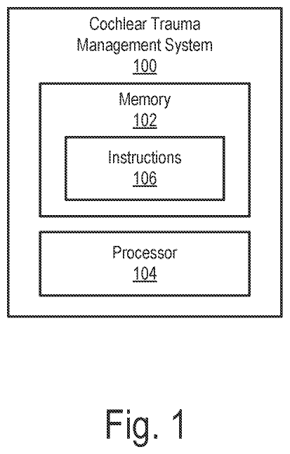

FIG. 1 shows an illustrative cochlear trauma management system 100 (“system 100”) configured to perform cochlear trauma management during an electrode lead insertion procedure. System 100 may be implemented in different ways and/or by different components of a cochlear implant system or device associated with a cochlear implant system (e.g., a mobile device communicatively coupled to the cochlear implant system, a clinician's fitting device, etc.). While certain examples described herein may focus on particular implementations of cochlear trauma management systems, it will be understood that it may be possible for other types of implementations to employ the principles being described (e.g., taking the place of the specific cochlear trauma management system implementations being described or operating in concert with those specific implementations).

System 100 may be implemented by computing resources such as an embedded computing device of a cochlear implant system. For example, computing resources embedded in a sound processor, a cochlear implant, a device coupled to the cochlear implant system (e.g., a mobile device such as a smartphone or music player, etc.), and/or another suitable device or system component may serve to perform the operations of system 100 as these operations are described herein. As will be described and illustrated in more detail below, certain implementations of system 100 may include only the computing resources (e.g., processors, memory, etc.) configured to perform operations described herein, while other implementations may further incorporate various other components of the cochlear implant system that relate to cochlear trauma management. For instance, certain implementations of system 100 may include the electrode lead being inserted, electrodes on the lead that are used to detect electrical potentials evoked in response to diagnostic tests, a loudspeaker configured to generate acoustic stimulation, storage facilities from which recipient attribute data may be accessed, input/output user interfaces, and/or any other suitable components described herein.

As illustrated in FIG. 1, this example implementation of system 100 includes, without limitation, a memory 102 and a processor 104 selectively and communicatively coupled to one another. Memory 102 and processor 104 may each include or be implemented by computer hardware that is configured to store and/or execute computer instructions (e.g., software, firmware, etc.). Various other components of computer hardware and/or software not explicitly shown in FIG. 1 may also be included within an implementation of system 100. In some examples, memory 102 and processor 104 may be distributed between multiple devices as may serve a particular implementation.

Memory 102 may store and/or otherwise maintain executable data used by processor 104 to perform any of the functionality described herein. For example, memory 102 may store instructions 106 that may be executed by processor 104. Memory 102 may be implemented by one or more memory or storage devices, including any memory or storage devices described herein, that are configured to store data in a transitory or non-transitory manner. Instructions 106 may be executed by processor 104 to cause system 100 to perform any of the functionality described herein. Instructions 106 may be implemented by any suitable application, software, firmware, script, code, and/or other executable data instance. Additionally, memory 102 may also maintain any other data accessed, managed, used, and/or transmitted by processor 104 in a particular implementation.

Processor 104 may be implemented by one or more computer processing devices, including general purpose processors (e.g., central processing units (CPUs), microprocessors, etc.), special purpose processors (e.g., application-specific integrated circuits (ASICs), field-programmable gate arrays (FPGAs), etc.), or the like. Using processor 104 (e.g., when processor 104 is directed to perform operations represented by instructions 106 stored in memory 102), system 100 may perform functions associated with intraoperative cochlear trauma management as described herein and/or as may serve a particular implementation.

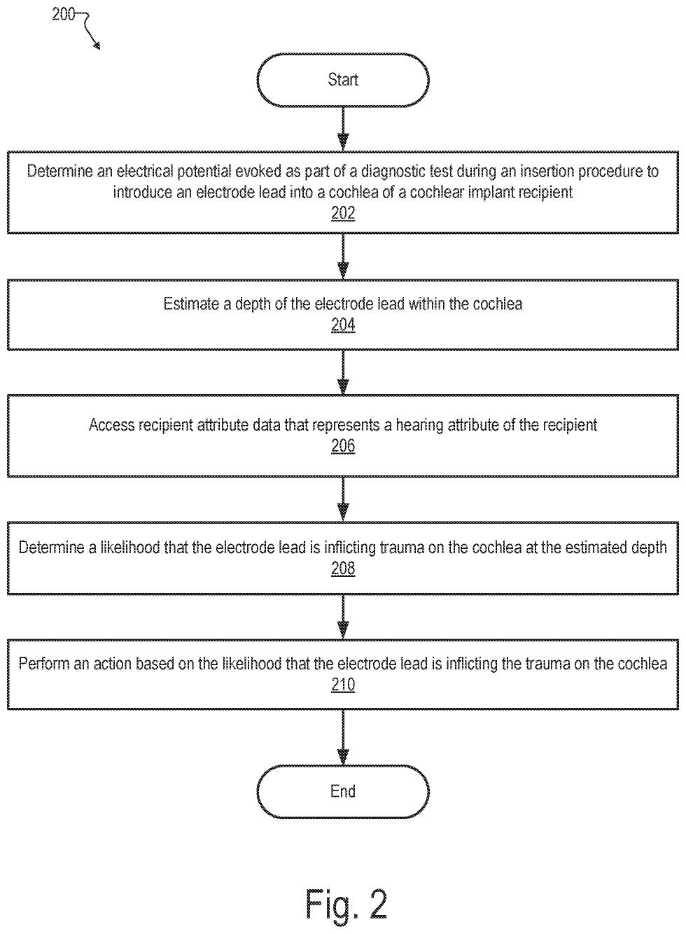

As one example of functionality that processor 104 may perform, FIG. 2 shows an illustrative method 200 for cochlear trauma management during an electrode lead insertion procedure. While FIG. 2 shows illustrative operations according to one embodiment, other embodiments may omit, add to, reorder, and/or modify any of the operations shown in FIG. 2. In some examples, multiple operations shown in FIG. 2 or described in relation to FIG. 2 may be performed concurrently (e.g., in parallel) with one another, rather than being performed sequentially as illustrated and/or described. One or more of the operations shown in FIG. 2 may be performed by a cochlear trauma management system such as system 100 and/or any implementation thereof. For instance, method 200 may be performed by a cochlear trauma management system implemented within a sound processor of a cochlear implant system, an external device coupled to a cochlear implant system, or another suitable system or device as may serve a particular implementation.

In some examples, the operations of FIG. 2 may be performed in real time so as to provide, receive, process, and/or use data described herein immediately as the data is generated, updated, changed, exchanged, or otherwise becomes available. Moreover, certain operations described herein may involve real-time data, real-time representations, real-time conditions, and/or other real-time circumstances. As used herein, “real time” will be understood to relate to data processing and/or other actions that are performed immediately, as well as conditions and/or circumstances that are accounted for as they exist in the moment when the processing or other actions are performed. For example, a real-time operation may refer to an operation that is performed immediately and without undue delay, even if it is not possible for there to be absolutely zero delay. Similarly, real-time data, real-time representations, real-time conditions, and so forth, will be understood to refer to data, representations, and conditions that relate to a present moment in time or a moment in time when decisions are being made and operations are being performed (e.g., even if after a short delay), such that the data, representations, conditions, and so forth are temporally relevant to the decisions being made and/or the operations being performed.

Each of operations 202-210 of method 200 will now be described in more detail as the operations may be performed by a cochlear trauma management system such as an implementation of system 100.

At operation 202, system 100 may determine an electrical potential evoked in response to acoustic stimulation applied to a recipient as part of a diagnostic test. For example, during an insertion procedure to introduce an electrode lead of a cochlear implant system into a cochlea of the recipient, system 100 may perform interoperative diagnostic tests by applying acoustic stimulation (e.g., one or more tones, etc.) to the recipient using a loudspeaker or other device capable of creating acoustic stimulation, and by then recording the electrical potentials that are evoked within the cochlea in response to the stimulation. These diagnostic tests may be any suitable type of intraoperative test configured to facilitate monitoring of cochlear health during the procedure. As one example, the diagnostic test that system 100 performs (and during which the electrical potential is evoked) may be an electrocochleography (ECochG) test in which the electrical potential is recorded, within the cochlea and using an electrode of the electrode lead, after the acoustic stimulation is applied. In some examples, diagnostic tests such as ECochG tests may be performed repeatedly and/or continuously during the procedure to monitor the health of the cochlea throughout the surgical procedure.

At operation 204, system 100 may estimate a depth of the electrode lead within the cochlea. This depth estimation may be made using any of a variety of depth estimation techniques that will be described in more detail below, or by a combination of multiple such techniques. The depth estimated at operation 204 may correspond to the electrical potential determined, at operation 202, to result from the diagnostic test. For example, the depth of the electrode lead may be estimated at approximately the same time that the electrical potential is recorded, or, if not estimated at the same time, the depth may at least be estimated before the electrode lead has moved significantly from the position where the diagnostic test was run. In this way, the test result (i.e., the recorded electrical potential) may be understood to correspond to the estimated depth. Additionally, as diagnostic tests may be performed repeatedly to monitor cochlear health in the ways mentioned above, it will be understood that cochlear lead depth may also be repeatedly and/or continuously estimated and monitored in a similar way (e.g., such that every recorded potential corresponds to its own estimated depth where the electrode lead was when the recording was made).

The electrode lead depth may be determined, stored, and expressed in any suitable way as may serve a particular implementation. For instance, the depth of the electrode lead may be considered to be the depth of a distal tip of the electrode lead into the cochlea with respect to a base of the cochlea (e.g., the round window where the electrode lead enters may be considered as a depth of zero, etc.). As another example, the depth may be based on how deep a particular recording electrode is located within the cochlea (e.g., past the round window or another such milestone). This recording electrode may be the distal-most electrode (the first electrode on the lead to enter the cochlea) or another suitable electrode that is proximal to one or more distal electrodes.

At operation 206, system 100 may access recipient attribute data that represents a hearing attribute of the recipient. The recipient attribute data may be generated based on preoperative analysis of the recipient. For instance, the recipient attribute data may represent an audiogram or other audiological report or analysis that has been performed prior to the surgical procedure (e.g., by a clinician overseeing care of the recipient). This audiological information may indicate how well the recipient is able to perceive different frequency components of sound, which, in turn, may reveal the distribution of signal generators (e.g., hair cells) along the cochlea (e.g., with respect to cochlear depth) that are still usable by the recipient for hearing sounds at certain frequencies. As another example, the recipient attribute data may indicate a specific etiology or mechanism of the recipient's hearing loss that may have been determined previously and that may have some bearing on what diagnostic test results can be expected for the particular recipient.

In both of these examples, the recipient attribute data may include data corresponding to various cochlear depths, or, in certain examples, data corresponding to the entirety of the cochlear chamber (e.g., the scala tympani, etc.) in which the electrode lead is being inserted. As such, the recipient attribute data may represent predictions, estimates, measurements, and/or other information corresponding specifically to the estimated depth of the electrode lead, such that the recipient attribute data may be used to “look up” what an electrical potential evoked from a diagnostic test may be expected to be for a given depth (e.g., the depth estimated at operation 204 where the diagnostic test was performed) under the given set of circumstances (e.g., based on the frequency or frequencies of the acoustic stimulation being applied for the diagnostic test and the corresponding characteristic frequency location or locations of that stimulation within the cochlea, etc.).

Consequently, at operation 208, system 100 may determine, based on the electrical potential determined at operation 202 and the recipient attribute data accessed at operation 206, a likelihood that the electrode lead is inflicting trauma on the cochlea at the depth estimated at operation 204. As will be described in more detail below, this determination may be made intraoperatively during the insertion procedure and using any of various techniques (or a combination of techniques) as may serve a particular implementation. A common thread with each of these techniques, however, is that system 100 analyzes the electrical potential that is recorded at a given depth for a given stimulus (e.g., with a tone at a frequency associated with a particular characteristic frequency location within the cochlea) not just in comparison to a general model of what electrical potential might be expected, but in comparison to a specific model that is customized for the particular recipient based on preoperative analysis that has already been performed. Accordingly, even if a general model may not predict that a certain result should be observed at a certain depth for a typical recipient, the customized model represented by the recipient attribute data may indicate that the anomalous result is likely to be observed at that depth for a particular recipient. As such, instead of incorrectly reporting cochlear trauma when the anomalous result is observed for the particular recipient (a false positive), system 100 is able to anticipate the anomaly, recognizing that it arises not from cochlear trauma but from idiosyncrasies of the specific recipient, and to avoid falsely reporting a high likelihood of imminent cochlear trauma.

At operation 210, system 100 may then perform an action based on the likelihood that the electrode lead is inflicting the trauma on the cochlea. For example, as will be described in more detail below, system 100 may do nothing if there is a relatively low likelihood of trauma, and, if there is a relatively high likelihood, system 100 may indicate as much to a human user (e.g., a surgeon or other surgical team member) and/or to a robotic system being used to perform the insertion procedure. For instance, the action may involve displaying an error message, sounding an audible alert, generating haptic feedback, directing a robotically-controlled instrument to change course (e.g., to slow, cease, or reverse the insertion of the lead, etc.), or any other action as may serve a particular implementation. As with other operations described above, operation 210 may be performed intraoperatively during the insertion procedure so that the surgeon and the recipient alike may benefit from this crucial information in real time while it may still be possible to mitigate long-term consequences of the cochlear trauma.

As mentioned above, while the cochlear trauma management system implemented by system 100 is shown to be implemented by computing resources such as memory 102 and processor 104, other cochlear trauma management system implementations may be integrated with, or may at least include certain components of, a cochlear implant system. For instance, a certain cochlear trauma management system implementation (referred to herein as an implementation of system 100) may include a cochlear implant configured to be implanted within a recipient; an electrode lead configured, upon being introduced into a cochlea of the recipient by way of an insertion procedure, to apply electrical stimulation to the recipient; a loudspeaker configured to apply acoustic stimulation to the recipient; and a processor configured to perform method 200 or other similar operations. In this example implementation, the processor may be configured to: 1) direct the cochlear implant to apply the electrical stimulation by way of the electrode lead and to direct the loudspeaker to apply the acoustic stimulation to the recipient as part of a diagnostic test; 2) determine, intraoperatively during the insertion procedure, an electrical potential evoked in response to the acoustic stimulation applied as part of the diagnostic test; 3) estimate a depth of the electrode lead within the cochlea, the estimated depth corresponding to the electrical potential resulting from the diagnostic test; 4) access recipient attribute data that represents a hearing attribute of the recipient, the recipient attribute data generated based on preoperative analysis of the recipient and including data corresponding to the estimated depth of the electrode lead; 5) determine, intraoperatively during the insertion procedure and based on the electrical potential and the recipient attribute data, a likelihood that the electrode lead is inflicting trauma on the cochlea at the estimated depth; and 6) perform, intraoperatively during the insertion procedure, an action based on the likelihood that the electrode lead is inflicting the trauma on the cochlea.

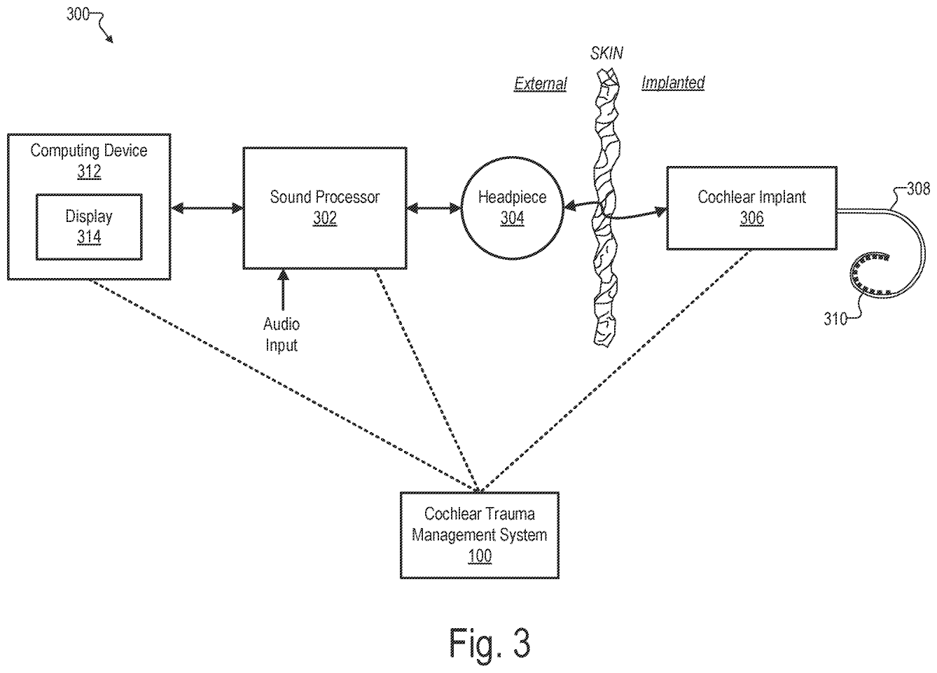

To illustrate how different implementations of system 100 may incorporate or interoperate with a cochlear implant system, FIG. 3 shows a configuration in which an illustrative cochlear implant system implements and/or interoperates with an illustrative implementation of system 100 to perform cochlear trauma management during an electrode lead insertion procedure.

More particularly, FIG. 3 shows illustrative aspects of a cochlear implant system 300 within which an implementation of a cochlear trauma management system such as system 100 may operate to perform operations such as those of method 200. While not explicitly shown in FIG. 3, it will be understood that cochlear implant system 300 may be used by a recipient within whom a cochlear implant and associated electrode lead is implanted. As shown, cochlear implant system 300 may include a sound processor 302 that receives one or more audio input signals (“Audio Input”), a headpiece 304 that transmits RF power and data through the skin, a cochlear implant 306 implanted within the recipient and receiving that power and data from headpiece 304, and an electrode lead 308 that has a plurality of electrodes 310 and that is introduced into a cochlea of the recipient by way of an insertion procedure. In some examples, cochlear implant system 300 may include or be communicatively coupled to a computing device 312 having a display 314.

As shown by dotted lines extending from an implementation of system 100 to various components of cochlear implant system 300 in FIG. 3, a cochlear trauma management system may be implemented by any of various computing devices in the configuration shown in FIG. 3. For example, system 100 may be implemented by computing resources in sound processor 302, cochlear implant 306, computing device 312, and/or by any combination of these computing resources and/or those of other suitable devices not explicitly shown in FIG. 3. As mentioned above, in certain examples several or all of these cochlear implant system components may be integrated together into a cochlear trauma management system.

Cochlear implant system 300 is shown in FIG. 3 to be a unilateral cochlear implant system (i.e., associated with only one ear of the recipient). Alternatively, a bilateral configuration of cochlear implant system 300 may include separate cochlear implants and electrode leads for each ear of the recipient. In the bilateral configuration, sound processor 302 may be implemented by a single processing unit configured to interface with both cochlear implants or by two separate processing units each configured to interface with a different one of the cochlear implants.

Cochlear implant 306 may be implemented by any suitable type of implantable stimulator. For example, cochlear implant 306 may be implemented by an implantable cochlear stimulator. Additionally or alternatively, cochlear implant 306 may be implemented by a brainstem implant and/or any other type of device that may be implanted within the recipient and configured to apply electrical stimulation to one or more stimulation sites located along an auditory pathway of the recipient.

In some examples, cochlear implant 306 may be configured to generate electrical stimulation representative of an audio signal (e.g., from the audio input) that is processed by sound processor 302 in accordance with one or more stimulation parameters transmitted to cochlear implant 306 by sound processor 302. Cochlear implant 306 may be further configured to apply the electrical stimulation to one or more stimulation sites (e.g., one or more intracochlear locations) within the recipient by way of one or more electrodes 310 on electrode lead 308. In some examples, cochlear implant 306 may include a plurality of independent current sources each associated with a channel defined by one or more of electrodes 310. In this manner, different stimulation current levels may be applied to multiple stimulation sites simultaneously by way of multiple electrodes 310.

Cochlear implant 306 may additionally or alternatively be configured to generate, store, and/or transmit data. For example, cochlear implant 306 may use one or more electrodes 310 to record one or more signals (e.g., one or more voltages, impedances, evoked responses within the recipient, and/or other measurements) and transmit, by way of a communication link (e.g., a same inductive link over which RF power is transmitted through the skin), data representative of the one or more signals to sound processor 302. This data may be referred to herein as back telemetry data.

Electrode lead 308 may be implemented in any suitable manner. For example, a distal portion of electrode lead 308 may be pre-curved such that electrode lead 308 conforms with the helical shape of the cochlea after being implanted. Electrode lead 308 may alternatively be naturally straight or of any other suitable configuration. In some examples, electrode lead 308 includes a plurality of wires (e.g., within an outer sheath) that conductively couple electrodes 310 to one or more current sources within cochlear implant 306. For example, if there are n electrodes 310 on electrode lead 308 and n current sources within cochlear implant 306, there may be n separate wires within electrode lead 308 that are configured to conductively connect each electrode 310 to a different one of the n current sources. Illustrative values for n may include 8, 12, 16, or any other suitable integer as may serve a particular implementation.

Electrodes 310 are located on at least a distal portion of electrode lead 308. In this configuration, after the distal portion of electrode lead 308 is inserted into the cochlea of the recipient, electrical stimulation may be applied by way of one or more of electrodes 310 to one or more intracochlear locations. One or more other electrodes (e.g., including a ground electrode, not explicitly shown) may also be disposed on other parts of electrode lead 308 (e.g., on a proximal portion of electrode lead 308) to, for example, provide a current return path for stimulation current applied by electrodes 310 and to remain external to the cochlea after the distal portion of electrode lead 308 is inserted into the cochlea. Additionally or alternatively, a housing of cochlear implant 306 may serve as a ground electrode for stimulation current applied by electrodes 310.

Sound processor 302 may be implemented by any type of processing unit configured to interface with (e.g., control and/or receive data from) cochlear implant 306. For example, sound processor 302 may transmit commands (e.g., stimulation parameters and/or other types of operating parameters in the form of data words included in a forward telemetry sequence) to cochlear implant 306 by way of an inductive link, provided by headpiece 304, through the recipient's skin. Along with this data, sound processor 302 may also provide RF power to cochlear implant 306 that the implant may use to operate and/or to charge a battery. In some examples, the data and power provided by sound processor 302 via headpiece 304 and the inductive link it creates with cochlear implant 306 may be combined to form an integrated forward telemetry signal. For example, the data words may be modulated onto RF power generated by sound processor 302. The transcutaneous link carrying data communications and/or RF power from sound processor 302 to cochlear implant 306 (as well as carrying backward telemetry communications in the other direction) may be implemented by any suitable number of wired and/or wireless bidirectional and/or unidirectional links. For example, the communication link may be implemented as an inductive link between coils within headpiece 304 and cochlear implant 306.

Sound processor 302 may be configured to perform various operations with respect to cochlear implant 306 (e.g., by executing instructions stored in memory within sound processor 302). For instance, sound processor 302 may be configured to control operation of cochlear implant 306 by receiving the audio input, processing the audio input in accordance with a sound processing program to generate appropriate stimulation parameters, and then transmitting the stimulation parameters to cochlear implant 306 (by way of headpiece 304) to direct cochlear implant 306 to apply electrical stimulation representative of the audio input to the recipient.

In some implementations, sound processor 302 may also be configured to apply acoustic stimulation to the recipient. For example, in an electroacoustic hearing system implementation of cochlear implant system 300, a loudspeaker (also referred to as an acoustic receiver) may be optionally coupled to sound processor 302 (not shown in FIG. 3). In this configuration, sound processor 302 may deliver acoustic stimulation to the recipient by way of the receiver. The acoustic stimulation may be representative of an audio signal configured to elicit an evoked response within the recipient. In examples in which sound processor 302 is configured to both deliver acoustic stimulation to the recipient and direct cochlear implant 306 to apply electrical stimulation to the recipient, cochlear implant system 300 may be referred to as an electroacoustic hearing system or another suitable term.

Sound processor 302 may be additionally or alternatively configured to receive and process data generated by cochlear implant 306. For example, sound processor 302 may receive data representative of a signal recorded by cochlear implant 306 using one or more electrodes 310 and, based on the data, may adjust one or more operating parameters of sound processor 302. Additionally or alternatively, sound processor 302 may use the data to perform one or more diagnostic operations with respect to cochlear implant 306 and/or the recipient. Other operations may be performed by sound processor 302 as may serve a particular implementation.

Sound processor 302 may be implemented by any suitable device that may be worn or carried by the recipient. For example, sound processor 302 may be implemented by a behind-the-ear (“BTE”) unit configured to be worn behind and/or on top of an ear of the recipient. Additionally or alternatively, sound processor 302 may be implemented by an off-the-ear unit (also referred to as a body worn device) configured to be worn or carried by the recipient away from the ear. In some examples, at least a portion of sound processor 302 may be implemented by circuitry within headpiece 304. In some cases, sound processor 302 and headpiece 304 may be fully integrated into a single device rather than as separate devices as shown in FIG. 3.

Headpiece 304 may be selectively and communicatively coupled to sound processor 302 by way of a communication link implemented by a cable or any other suitable wired or wireless communication link. Headpiece 304 may be implemented in any suitable manner to facilitate communication between sound processor 302 and cochlear implant 306. For instance, headpiece 304 may include an external antenna (e.g., a coil and/or one or more wireless communication components) configured to facilitate selective wireless coupling of sound processor 302 to cochlear implant 306. Headpiece 304 may additionally or alternatively be used to selectively and wirelessly couple any other external device to cochlear implant 306. To this end, headpiece 304 may be configured to be affixed to the recipient's head (e.g., by way of a magnet, a hair clip, etc.) and positioned such that the external antenna housed within headpiece 304 becomes aligned with a corresponding implantable antenna (which may also be implemented by a coil and/or one or more wireless communication components) included within or otherwise connected to cochlear implant 306. In this manner, communicative lock between sound processor 302 and cochlear implant 306 may be achieved and stimulation parameters and/or RF power may be wirelessly and transcutaneously transmitted between sound processor 302 and cochlear implant 306 by way of headpiece 304.

The audio input may provide, to sound processor 302, an audio signal representative of audio content to be conveyed to the recipient. As described above, sound processor 302 may communicate data representative of this audio signal to cochlear implant 306 by way of headpiece 304. In this way, sound processor 302 may direct cochlear implant 306 to apply electrical stimulation representative of the audio signal to the recipient (e.g., by way of current applied via electrodes 310 on electrode lead 308, as described above). The audio signal provided by the audio input may include or otherwise be representative of any suitable audio content that is to be conveyed to the recipient. For instance, the audio input may include microphone input from a microphone included within cochlear implant system 300 and worn or carried by the recipient (e.g., a microphone configured to be placed within the concha of the ear near the entrance to the ear canal, such as a T-MIC™ microphone from Advanced Bionics, a microphone held within the concha of the ear near the entrance of the ear canal during normal operation by a boom or stalk that is attached to an ear hook configured to be selectively attached to sound processor 302, etc.), from a microphone located apart from the recipient and communicatively coupled to cochlear implant system 300 (e.g., placed near a person speaking at a conference, worn by a companion of the recipient in a noisy restaurant, etc.), or any other suitable microphone or set of microphones placed at any suitable location as may serve a particular implementation. As another example, the audio input may include an audio source of a recording (e.g., music, a spoken word recording, etc.) or another audio file or stream.

Computing device 312 may be configured to communicatively couple to sound processor 302 by way of any suitable wired or wireless communication link. Computing device 312 may be implemented by any suitable combination of hardware and software. For example, computing device 312 may be implemented by a mobile device (e.g., a mobile phone, a laptop, a tablet computer, etc.), a desktop computer, and/or any other suitable computing device as may serve a particular implementation. In certain implementations, computing device 312 may be implemented by a mobile device configured to execute an application (e.g., a “mobile app”) that may be used by a user (e.g., the recipient, a clinician, and/or any other user) to control one or more settings of sound processor 302 and/or cochlear implant 306. Computing device 312 may perform one or more operations (e.g., diagnostic operations) with respect to data generated by sound processor 302 and/or cochlear implant 306.

In some examples, computing device 312 may be configured to control an operation of cochlear implant 306 by transmitting one or more commands to cochlear implant 306 by way of sound processor 302. Likewise, computing device 312 may be configured to receive data generated by cochlear implant 306 by way of sound processor 302. Alternatively, computing device 312 may interface with (e.g., control and/or receive data from) cochlear implant 306 directly by way of a wireless communication link between computing device 312 and cochlear implant 306. In some implementations in which computing device 312 interfaces directly with cochlear implant 306, sound processor 302 may or may not be included in cochlear implant system 300.

Computing device 312 is shown as having an integrated display 314. Display 314 may be implemented by a display screen, for example, and may be configured to display content generated by computing device 312. Additionally or alternatively, computing device 312 may be communicatively coupled to an external display device (not shown) configured to display the content generated by computing device 312.

In some examples, computing device 312 represents a fitting device configured to be selectively used (e.g., by a clinician) to fit sound processor 302 and/or cochlear implant 306 to the recipient. In these examples, computing device 312 may be configured to execute a fitting program configured to set one or more operating parameters of sound processor 302 and/or cochlear implant 306 to values that are optimized for the recipient. As such, in these examples, computing device 312 may not be considered to be part of cochlear implant system 300. Instead, computing device 312 may be considered to be separate from cochlear implant system 300 such that computing device 312 may be selectively coupled to cochlear implant system 300 when it is desired to fit sound processor 302 and/or cochlear implant 306 to the recipient.

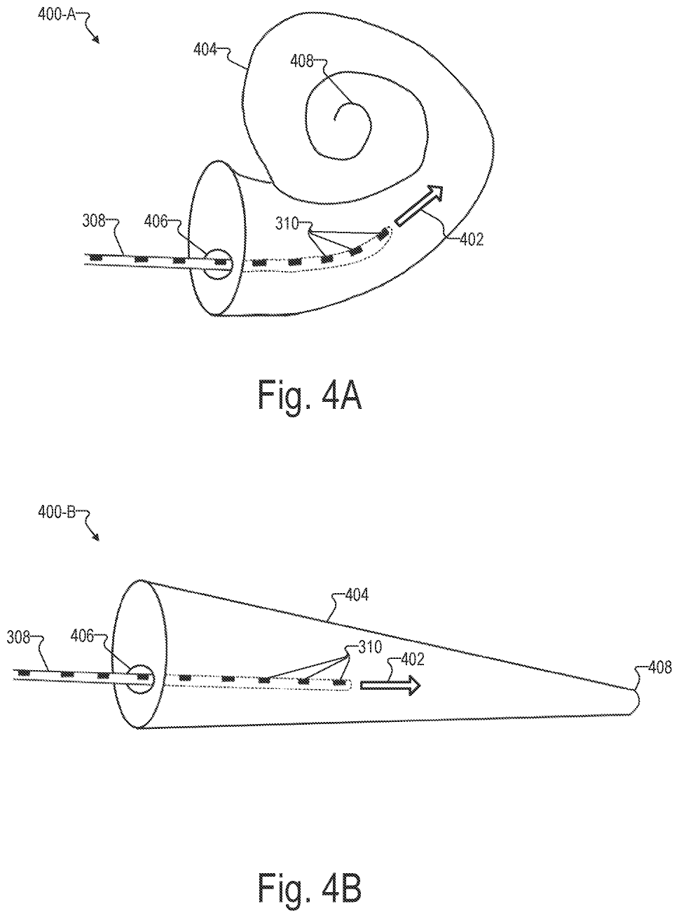

FIGS. 4A-4B show different views of a cochlea of a cochlear implant recipient during an illustrative electrode lead insertion procedure. Specifically, FIG. 4A shows a first view 400-A in which an insertion procedure 402 is performed to introduce electrode lead 308 (described above) into a cochlea 404 of a recipient. As shown, electrodes 310 are included on electrode lead 308, and insertion procedure 402 involves introducing electrode lead 308 into cochlea 404 through an entry point 406 (e.g., the round window of cochlea 404, the oval window of cochlea 404, etc.) and curving the lead, following the spiral shape of cochlea 404, as the lead is inserted toward an apex 408 of cochlea 404. The curvature, size, and delicateness of cochlea 404, as well as the limited visibility and status feedback available to the surgical team, make insertion procedure 402 a difficult and delicate surgical procedure in which it is possible for cochlear trauma to occur (e.g., if electrode lead 308 runs up against or even penetrates through the walls of the cochlear duct it is being introduced into). As such, systems and methods for cochlear trauma management are described herein to facilitate highly effective monitoring of cochlear health during such an electrode lead insertion procedure such as insertion procedure 402.

View 400-B of FIG. 4B is shown to include all the same features as described above for view 400-A, but, in this case, cochlea 404 is shown to be “unrolled” for convenience of illustration. Specifically, with the straightened-out representation of cochlea 404, the depth of electrode lead 308 within cochlea 404 is more readily apparent from the drawing so as to be clearly perceived and described. Insertion procedure 402 is shown in this view to involve inserting electrode lead 308 from entry point 406 toward apex 408 in a straight line; accordingly, the depth of the electrode lead can be understood to be a position along a horizontal axis extending from entry point 406 to apex 408. While other FIGS. below will utilize this “unrolled” representation of view 400-B for illustrative convenience, it will be understood that insertion procedures described herein may actually be performed on human cochleae that curve in the manner shown by view 400-A.

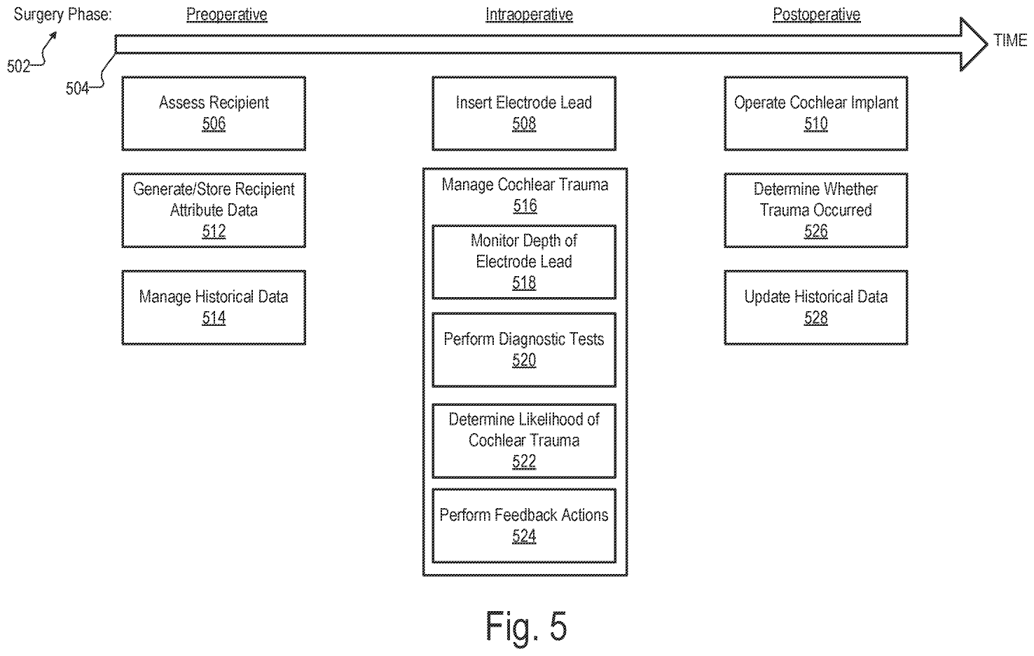

FIG. 5 shows illustrative actions that a cochlear trauma management system such as system 100 may perform at different surgery phases 502 before, during, and after an electrode lead insertion procedure. Specifically, FIG. 5 shows a preoperative phase including actions to be performed prior to the electrode lead insertion procedure, an intraoperative phase including actions to be performed during the electrode lead insertion procedure, and a postoperative phase including actions to be performed subsequent to the electrode lead insertion procedure. As shown, actions to be performed during each of these three surgery phases 502 are laid out in FIG. 5 along a timeline 504. For example, an action 506 involving assessing the recipient is laid out under the preoperative phase, an action 508 involving inserting the electrode lead into the cochlea is laid out under the intraoperative phase, and an action 510 involving normal operation of the cochlear implant system is laid out under the postoperative phase.

Actions 506-510 are typical actions performed for any cochlear implant system recipient. For example, an assessment of a person suffering from hearing loss may indicate that the person is a viable candidate for a cochlear implant system (action 506), the person receives the cochlear implant system by being surgically implanted with a cochlear implant (and its corresponding electrode lead) and having the cochlear implant system fitted to their specific needs/preferences (action 508), and the person may then use the cochlear implant system in their everyday life (action 510).

As shown in FIG. 5, other actions 512-528 may be performed along with these conventional actions to implement the cochlear trauma management described herein. To be specific, preoperatively, an action 512 may involve generating and storing recipient attribute data collected as part of the recipient assessment of action 506, and an action 514 may involve managing historical data collected from other similar surgical procedures performed on other recipients (or for the other cochlea of the present recipient). Intraoperatively, an action 516 may involve managing cochlear trauma by performing actions such as those described in relation to method 200. For instance, action 516 is shown to involve an action 518 for monitoring the depth of the electrode lead being inserted, an action 520 for performing diagnostic tests at different depths (e.g., ECochG tests or other such intraoperative acoustic-based diagnostics), an action 522 for determining the likelihood of cochlear trauma based on the diagnostic testing and insight from preoperatively-generated models, and an action 524 for indicating or acting on the determined likelihood of trauma by performing one or more feedback actions. Postoperatively, an action 526 may involve determining whether cochlear trauma actually did occur during the insertion procedure, and an action 528 may involve updating the historical data to allow future insertion procedures to benefit from what can be learned from this insertion procedure (e.g., including what trauma was predicted, on what basis it was predicted, the ultimate accuracy/validity that such predictions turned out to have, etc.).

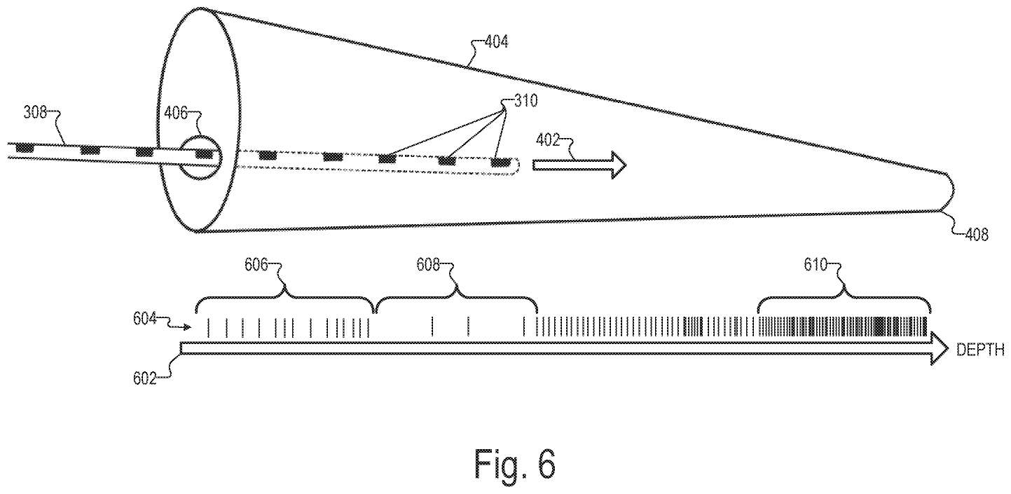

Each of actions 512-528 will now be described in more detail. For this description, certain principles and examples are illustrated in FIGS. 6-13. Accordingly, the following description makes reference to FIGS. 6-13, along with references to FIG. 5, to illustrate how cochlear trauma management systems may perform actions 512-528.

Action 512 may be performed by generating and/or storing recipient attribute data that is determined preoperatively, such as while the recipient is being assessed at action 506. Recipient attribute data generated and/or stored by action 512 may represent various characteristics of the recipient that may be pertinent to results that are to be expected from diagnostic tests performed at different depths during the insertion procedure. As one example, the recipient attribute data may indicate a signal generator profile along the cochlea of the recipient. As used herein, signal generators along the cochlea refer to hair cells or other sources of electrical potentials that are recorded as evoked responses to acoustic or other stimulation during diagnostic tests (e.g., ECochG tests or the like). Accordingly, signal generator profiles refer to distributions of such signal generators along the cochlea, which may be particular to each individual and may hence vary from recipient to recipient. For example, one recipient may retain a number of functional signal generators near an apex of his or her cochlea even while lacking signal generators closer to the base of the cochlea, thereby enabling this recipient to naturally hear certain low-pitched frequencies while suffering significant hearing loss with respect to high-pitched frequencies. In contrast, a different recipient may retain signal generators near the base of the cochlea while lacking functional signal generators nearer to the apex. In this case then, this recipient may retain an ability to naturally perceive higher-pitched frequencies while struggling to hear lower-pitched frequencies.

Along these lines, each recipient may have his or her own signal generator profile (indicating certain frequencies that are easy to naturally perceive and other frequencies that are difficult or impossible to perceive) that is based on idiosyncrasies unique to the recipient and that is represented in recipient attribute data generated and/or stored by way of action 512. The signal generator profile for a given recipient may be determined in any suitable way. For instance, the profile may be determined as part of (or be otherwise associated with) a preoperative audiogram performed for the recipient prior to the insertion procedure. An audiogram may be created during a clinical session in which different tones are presented to a recipient to test how well the recipient can perceive sound at various frequencies. Based on such testing, the audiogram may indicate the profile of which frequencies are perceivable by the recipient and, as a result of the direct relationship between sound frequency and cochlear depth, a signal generator profile may be created based on the audiogram.

Another example of recipient attribute data that may be generated and/or stored as part of action 512 is etiological data representative of a mechanism by which the recipient experiences hearing loss (e.g., the mechanism of hearing loss that is particular to the recipient). Different mechanisms of hearing loss may affect diagnostic testing such as ECochG testing differently, and, as such, it may be useful to account for such etiological data when generating a model of diagnostic test results that are to be expected. Such etiological data may be determined based not only on the audiogram described above but also on other preoperative measures such as how a recipient's sensitivity changes as a sound level changes, what otoacoustic emissions are generated by the recipient as sound levels change, psychoacoustic measures of cochlear compression, and so forth.

FIG. 6 shows illustrative aspects of recipient attribute data that may represent hearing attributes of a particular recipient and may be determined and stored at action 512 (as well as accessed later to facilitate cochlear trauma management in ways described in more detail below). As shown, FIG. 6 depicts the insertion procedure 402 of electrode lead 308 that was described above in relation to FIG. 4B using the unrolled representation of cochlea 404. Beneath the representation of cochlea 404, FIG. 6 also shows a depth axis 602 along which a signal generator profile 604 is illustrated. More particularly, small lines representative of signal generators are depicted along depth axis 602 to show where (i.e., at which frequencies/cochlear depths) this particular recipient may retain some ability to naturally perceive sound and where the recipient may struggle or lack any such perception.

In this example, for instance, FIG. 6 calls out a region 606 near the base of cochlea 404 (e.g., near entry point 406 where electrode lead 308 enters cochlea 404) that is shown to include a relatively sparse distribution of signal generators (such that the recipient may retain limited hearing at these high frequencies), a region 608 that is a little further into cochlea 404 and includes an extremely sparse distribution of signal generators (such that the recipient likely retains little or no hearing ability at these frequencies), and a region 610 near apex 408 of cochlea 404 that features a relatively dense distribution of signal generators (such that the recipient may retain relatively normal hearing at these low frequencies). Other regions not explicitly called out in FIG. 6 are shown to have their own distribution densities such that the overall signal generator profile comprises a relatively complex and unique representation of idiosyncratic hearing abilities of this specific recipient. As has been described, this profile may be determined and represented in recipient attribute data in any suitable way so as to be utilized for cochlear trauma management operations described herein.

Returning to FIG. 5, action 514 may also be performed (along with action 512) prior to the electrode lead insertion procedure for this particular recipient. While the recipient attribute data of action 512 may include data specifically related to characteristics of the recipient herself, action 514 may involve managing historical data related to other recipients (as well as to another cochlea of this particular recipient) who have been surgically operated on in previous insertion procedures. As will be described in more detail below, this data may be used by the cochlear trauma management system to calibrate and learn (e.g., via machine learning or other such processes) how to most accurately interpret diagnostic test results. For example, the historical data may be associated with a set of electrical potentials recorded during a plurality of previous insertion procedures that include both 1) a first subset of insertion procedures determined to have resulted in cochlear trauma, and 2) a second subset of insertion procedures determined to have resulted in no cochlear trauma. By analyzing distinctions between diagnostic test results recorded in these traumatic and atraumatic insertion procedures, the cochlear trauma management system may more accurately interpret intraoperative test results being received in real time, as will be described in more detail below.

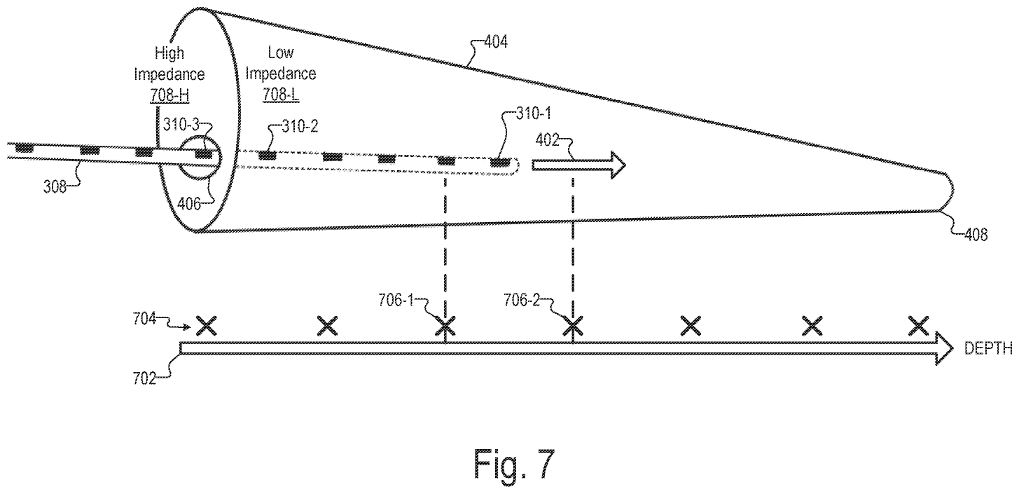

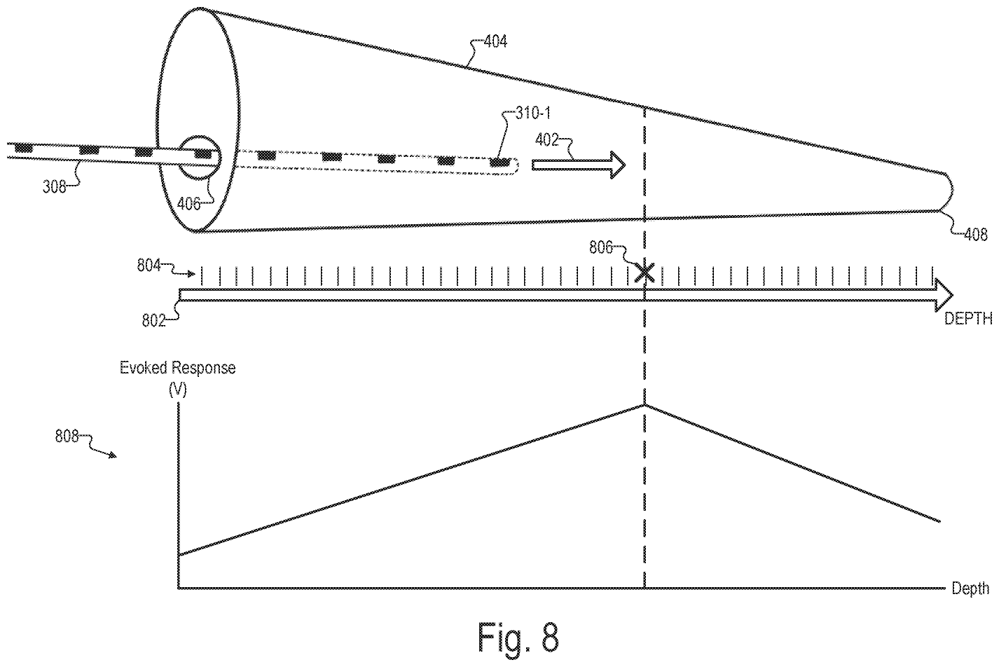

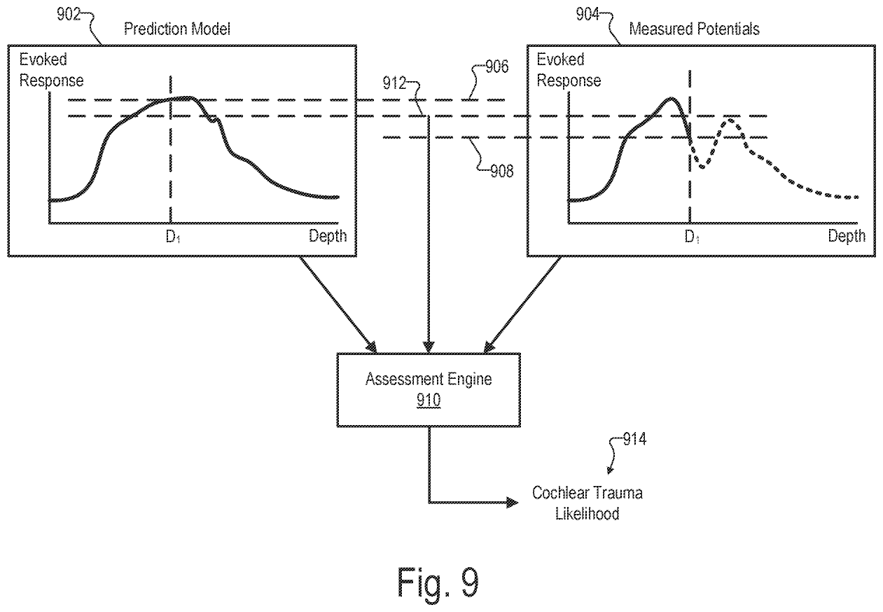

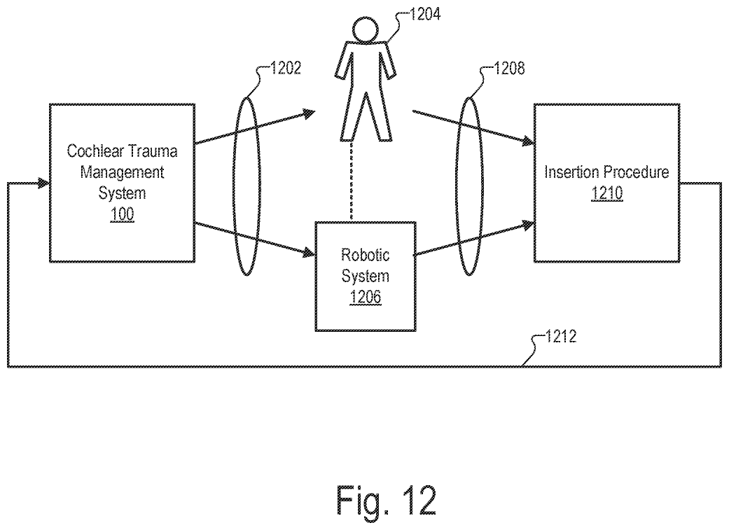

Moving on now to action 516, FIGS. 7-12 will be described to show examples of how the cochlear trauma management system may intraoperatively manage cochlear trauma at action 516 by way of actions 518-524. Specifically, FIG. 7 shows illustrative aspects of various ways a depth of the electrode lead may be estimated during the electrode lead insertion procedure to implement action 518; FIG. 8 shows illustrative aspects of how diagnostic tests may be performed during an electrode lead insertion procedure to implement action 520; FIGS. 9-11 show various illustrative ways that measured electrical potentials and recipient attribute data may be used to intraoperatively determine a likelihood that an electrode lead is inflicting trauma on a cochlea at a particular depth pursuant to action 522; and FIG. 12 shows illustrative aspects of how one or more feedback actions 524 may be performed by the cochlear trauma management system to ensure that the insertion procedure is effective and successful.

As illustrated by FIG. 7, one or more of various techniques may be used to intraoperatively monitor the cochlear depth of electrode lead 308. For example, a first technique may involve applying multi-tone stimulation to the recipient (a “multi-tone technique”). If the characteristic frequency locations of these tones are distributed at various depths along the path of the insertion, the cochlear trauma management system may monitor the depth of a particular recording electrode as the electrode is detected to approach and pass each of the various depths associated with the different tones. A second technique may involve stimulating multiple electrodes along the lead and using the difference of impedance on either side of the round window (i.e., outside and inside the cochlea) to estimate the number of electrodes that have entered the cochlea, and correspondingly how far past the entry point each of the electrode may be inferred to be (an “impedance-based technique”). A third technique may rely on a measured passage of time to estimate the depth based on certain assumptions about the rate of insertion and a point in time when the depth is known or can be reliably estimated (a “time-based technique”). While none of these techniques may be sure to precisely determine a perfectly accurate depth under all circumstances, each of these and/or other techniques may be used alone or in combination with one another to effectively estimate and monitor the insertion procedure progress as the depth of the electrode lead increases.

To illustrate, FIG. 7, similar to FIG. 6, is shown to depict the insertion procedure 402 of electrode lead 308 using the unrolled representation of cochlea 404. Beneath the representation of cochlea 404, FIG. 7 shows a depth axis 702 similar to depth axis 602 described above. However, rather than showing the signal generator profile 604 that was described in relation to FIG. 6, FIG. 7 depicts various characteristic depths 704 that correspond to tones of a multi-tone stimulus used to implement the multi-tone technique described above. As shown, the tones of this stimulus may be selected such that characteristic depths 704 are relatively evenly distributed along the length of the cochlea duct and the cochlear trauma management system may therefore estimate the depth of electrode lead 308 based on which characteristic depths 704 a particular recording electrode 310-1 (e.g., a distal electrode as shown, or another suitable electrode 310) is detected to have passed.

More particularly, this first technique for estimating the depth of electrode lead 308 within cochlea 404 may include, for example: 1) applying the acoustic stimulation as multi-tone stimulation including a first component at a first frequency associated with a first depth 706-1 within cochlea 404 and a second component at a second frequency associated with a second depth 706-2 within cochlea 404; 2) determining, based on an electrical potential evoked in response to the acoustic stimulation, that recording electrode 310-1 (i.e., the electrode used to record the electrical potential) has passed first depth 706-1 and has yet to pass second depth 706-2; and 3) estimating the depth of the electrode lead based on this determination that electrode 310-1 has passed first depth 706-1 and has yet to pass second depth 706-2. The determination that recording electrode 310-1 has passed a particular depth may be made in any suitable way. For instance, for acoustic-based diagnostic tests (e.g., ECochG tests, etc.) that are repeatedly performed based on a tone with a frequency associated with the particular depth (referred to as a “characteristic depth”), the determination may be made based on an observation that evoked responses of the tests rise as the recording electrode approaches the particular depth and then, when the recording electrode passes the particular depth, an observation that the evoked response begin to fall, change phase, or otherwise react to the passing of the characteristic depth in a predictable way. By performing this analysis for each characteristic depth 704 associated with one of the tones used for the multi-tone stimulation, the depth of recording electrode 310-1 (and hence of electrode lead 308) may be estimated to a level of accuracy governed by how close the tones are to one another (i.e., how closely the characteristic depths 704 are spaced).

To illustrate the impedance-based technique mentioned above, FIG. 7 also indicates that a low impedance 708-L may be present inside cochlea 404 (e.g., detectable by electrodes that have passed through entry point 406) while a high impedance 708-H may be present outside of cochlea 404 (e.g., detectable by electrodes that have yet to pass through entry point 406). This is due to the presence of fluid within the cochlea that helps sound waves propagate through the cochlear chamber and the presence of air (which has a higher electrical impedance than the fluid) outside of the cochlea.

This impedance-based technique for estimating the depth of electrode lead 308 within cochlea 404 may include, for example: 1) detecting a first impedance of a first electrode (e.g., electrode 310-2) included on the electrode lead; 2) detecting a second impedance of a second electrode (e.g., electrode 310-3) included on the electrode lead (where, as shown by electrodes 310-2 and 310-3, the second electrode is adjacent to the first electrode so as to follow the first electrode into the cochlea as the electrode lead is introduced into the cochlea during the insertion procedure); 3) determining, based on the first and second impedances, that the first electrode has passed through a round window (or other entry point 406) of cochlea 404 and that the second electrode has yet to pass through the round window; and 4) estimating the depth of the electrode lead based on the determining that the first electrode has passed through the round window and that the second electrode has yet to pass through the round window. In the illustrated example, by detecting low impedance 708-L for electrode 310-2 while detecting high impedance 708-H for electrode 310-3, the cochlear trauma management system may infer that electrode 310-2 (as well as the four more distal electrodes 310 in front of it) have passed through entry point 406 into the cochlea while electrode 310-3 is still on the other side of entry point 406. The depth of electrode lead 308 may be directly determined based on this inference, accurate to within the distance between electrodes 310-2 and 310-3.

The time-based depth detection technique mentioned above to rely on elapsed time may be used alone or in connection with either or both of the other depth detection techniques that have been described. Specifically, for example, this technique may include estimating the depth electrode lead 308 within cochlea 404 by: 1) determining an amount of time that has elapsed during the insertion procedure since the electrode lead was at a known depth (e.g., since the electrode lead first breached cochlea 404 at entry point 406, etc.); and 2) estimating the depth of the electrode lead based on the known depth and the amount of time that has elapsed during the insertion procedure since the electrode lead was at the known depth. This time-based technique presumes that insertion procedure 402 proceeds at a known and relatively constant insertion rate, such that, given an amount of time that has passed since the electrode lead was at the known depth, an up-to-date depth may be tracked as time passes. The accuracy of depth estimates made using this technique will be governed by the extent to which this presumption reflects reality in a given situation. That is, time may serve as a good proxy for depth to the extent that the insertion procedure proceeds at a set and consistent rate that is well understood, while time may fail to closely mirror depth if the rate is inconsistent or different (e.g., faster or slower, more erratic and changing, etc.) than it is presumed to be.

As mentioned above, FIG. 8 shows illustrative aspects of how diagnostic tests may be performed during an electrode lead insertion procedure to implement action 520. Similar to FIGS. 6 and 7, FIG. 8 shows the ongoing insertion procedure 402 being performed to insert electrode lead 308 into the unrolled representation of cochlea 404, and a depth axis 802 is extended out below the representation of the cochlea. In this case, various testing depths 804 are illustrated along depth axis 802 to indicate locations where a diagnostic test such as an ECochG test may be performed. Additionally, a characteristic depth 806 is shown that will be understood to correspond to a tone of stimulation being applied for each of these tests. In this example, a single fixed tone is shown to be applied, though, as described above, it will be understood that certain examples may utilize stimulation having multiple tones (or, in some cases, non-fixed tones that change in predetermined ways).

FIG. 8 also shows a graph 808 that is aligned with the cochlea representation and depth axis 802 to illustrate depth along its x-axis. Graph 808 plots an illustrative evoked response (e.g., an electrical potential measure in volts (“V”)) for each of the tests performed at the testing depths 804. It will be understood that this plot is a general and idealized plot to illustrate the increasing of the evoked response during the approach until characteristic depth 806 is reached (i.e., the depth associated with the tone being used in the acoustic stimulation to evoke the electrical potentials) and the falling off of the evoked response after characteristic depth 806 is passed. This principle was described above in relation to the multi-tone depth detection technique and will be understood to generally hold true under many circumstances though, as will be shown in more detail below, the exact shape of this curve for a particular recipient will be dependent on various factors including the distribution of signal generators, whether trauma occurs, and so forth.

ECochG tests or other diagnostic testing performed at each testing depth 804 may be performed in any manner as may serve a particular implementation. For example, a loudspeaker may generate the stimulus as a tone burst having a particular frequency (e.g., 500 Hz), a particular duration (e.g., 50 ms), and a particular intensity or volume (e.g., 100 dB SPL). Several times per second, ECochG response may then be recorded by recording electrode 310-1 as electrode lead continues moving forward as part of insertion procedure 402. As mentioned above, each testing depth 804 may be correlated with the recorded electrical potential (i.e., the evoked response indicated for that depth by graph 808) measured for that depth.

Returning to FIG. 5, action 522 may be performed by analyzing whatever the electrical potential for a particular depth was actually recorded to be in comparison to what the recipient attribute data indicates the electrical potential is expected to be (e.g., either if there is or is not cochlear trauma occurring). In other words, given the electrical potential and estimated depth that have been determined at actions 518 and 520, action 522 involves accessing or creating a computational auditory model (e.g., based on recipient attribute data that has been accessed after being generated and stored preoperatively at action 512) that can be utilized to determine the likelihood that cochlear trauma is being inflicted. This determination of action 522 may be based on any of various factors that will now be described, or may be based on a combination of such factors.

A first factor that may be taken into account while assessing the likelihood is the extent to which recorded electrical potentials align with predicted electrical potentials or whether they differ by more than a predetermined threshold. FIG. 9 illustrates how this “threshold factor” may be taken into account for an illustrative prediction model (generated based on recipient attribute data generated prior to the insertion procedure) and for illustrative evoked response (measured electrical potentials recorded during the insertion procedure in response to diagnostic tests such as described in FIG. 8).

More particularly, FIG. 9 shows a prediction model 902 that is customized for a particular cochlea of a particular recipient by being based on recipient attribute data generated from the types of preoperative analysis that have been described (e.g., audiograms for determining a signal generator profile, etiological tests involving detecting otoacoustic emissions, etc.). Prediction model 902 will be understood to represent a custom prediction of what evoked response data (e.g., electrical potentials recorded in response to ECochG tests performed with a particular stimulus) will be detected, as a function of cochlear depth, if no trauma occurs as the electrode lead is inserted. As shown, a basic shape similar to graph 808 is represented (i.e., the evoked response goes up until a particular characteristic depth associated with the stimulation is reached, then goes down after passing that depth), but there is more nuance and detail in the prediction model due to characteristics (e.g., idiosyncrasies) of this particular recipient that are being accounted for.

FIG. 9 also shows a parallel graph of measured potentials 904 alongside prediction model 902. While the entire predicted model may be known as the insertion procedure begins (since it is based on preoperative recipient attribute data), measured potentials 904 are based on real-time test results being performed during the insertion procedure. As such, it will be understood that the dotted portion of the curve beyond a depth D₁ is not yet known at a time when the insertion procedure has advanced to depth D₁. D₁ will be understood to represent an arbitrary depth that the electrode lead has been inserted to at a particular point in time during the insertion procedure when this example is occurring. D₁ may be determined and correlated to the measured electrical potentials in any of the ways described above.

For this example, when the electrode lead is at depth D₁, prediction model 902 may indicate that the electrical potential at this depth is predicted to be at a response level 906, while measured potentials 904 indicates that the electrical potential actually recorded at this depth is at a response level 908. Taking both of these response levels into account, an assessment engine 910 (e.g., comparison hardware and/or software included within the cochlear trauma management system) may compare the predicted response level 906 and the recorded response level 908 with respect to a threshold level 912. In this case, threshold level 912 is set a little below the predicted response level 906 to reflect that the measured potential may diverge slightly from the prediction without necessarily being likely to represent cochlear trauma. When a measured potential is recorded lower than threshold level 912, however, assessment engine 910 may be configured to interpret that as a likelihood that cochlear trauma is being inflicted. This likelihood is output by assessment engine 910 as cochlear trauma likelihood 914.

Accordingly, as illustrated in FIG. 9, the determining of cochlear trauma likelihood 914 is performed (for action 522) by: 1) determining, based on accessed recipient attribute data, prediction model 902 for the electrical potential at the estimated depth D₁ in an absence of cochlear trauma; 2) determining, based on prediction model 902, a threshold level 912 that the electrical potential is predicted to exceed for the estimated depth in the absence of cochlear trauma; and 3) determining likelihood 914 based on whether the electrical potential (i.e., the measured potential 904 recorded at depth D₁ to be response level 908) exceeds threshold level 912. Since, in this example, response level 908 does not exceed threshold level 912, likelihood 914 may be set to indicate that cochlear trauma is likely being inflicted. Conversely, for depths where response level 908 more closely aligns with response level 906 (e.g., and does not fall below respective threshold levels set for those depths), likelihood 914 would be set to indicate that cochlear trauma is not likely being inflicted.

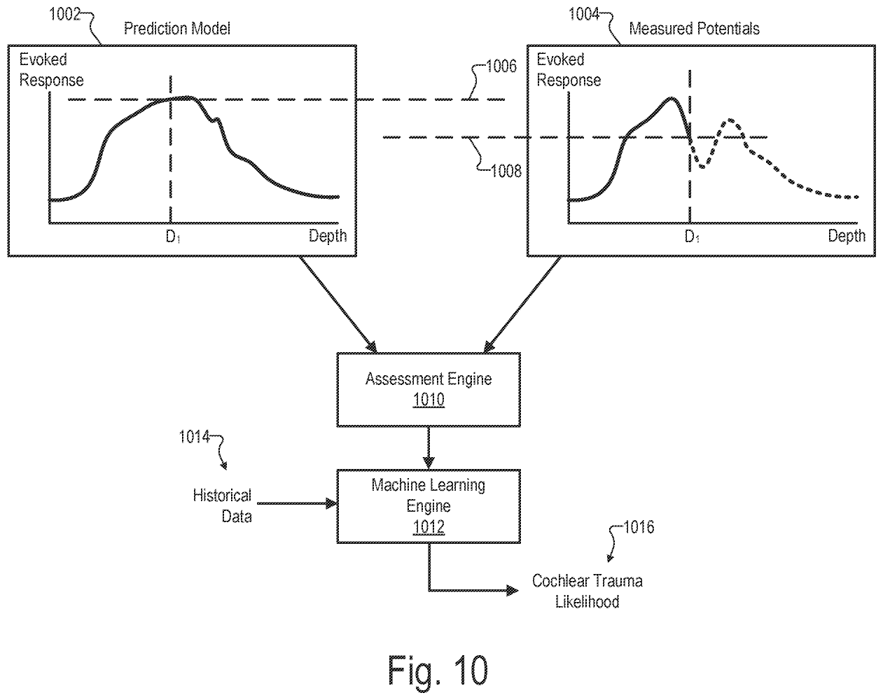

A second factor that may be taken into account while assessing the likelihood is the extent to which historical data from other insertion procedures (e.g., the historical data managed at action 514) suggests that the recorded feedback is likely to correspond to cochlear trauma. For instance, the comparison between historical data and presently-recorded feedback may be performed using machine learning, deep neural networks, or another such technologies. FIG. 10 illustrates how this “machine learning factor” may be taken into account for an illustrative prediction model and illustrative measured potentials (similar to prediction model 902 and measured potentials 904 described above).

More particularly, FIG. 10 shows a prediction model 1002 that is customized for a particular cochlea of a particular recipient and, similar to prediction model 902, represents a custom prediction of what evoked response data will be detected, as a function of cochlear depth, if no trauma occurs as the electrode lead is inserted. FIG. 10 also shows a parallel graph of measured potentials 1004 and a particular depth D₁ that is selected as an arbitrary depth for purposes of this example. Similar to the example of FIG. 9, when the electrode lead is at depth D₁ for this example, prediction model 1002 may indicate that the electrical potential at this depth is predicted to be at a response level 1006, while the measured potentials 1004 graph indicates that the electrical potential actually recorded at this depth is at a response level 1008.

An assessment engine 1010 may take both of these levels into account (e.g., performing a hardware or software comparison to analyze the difference between the levels, etc.), but, rather than (or in addition to) comparing the predicted response level 906 and the recorded response level 908 with respect to a threshold level as performed for the “threshold factor” described in FIG. 9, a machine learning engine 1012 may instead be used in this example. Specifically, machine learning engine 1012 (e.g., another software or hardware process included in the cochlear trauma management system) is shown to input data from assessment engine 1010 (indicating how the levels compare) as well as to input historical data 1014 (indicating how levels have compared in similar circumstances for other insertion procedures that may or may not have turned out to inflict trauma). Using principles of machine learning, artificial intelligence, and other such technologies, machine learning engine 1012 may determine whether the way response levels 1006 and 1008 are behaving appears to look more like historical data for procedures that did result in trauma or historical data for procedures that did not result in trauma. This likelihood is output as cochlear trauma likelihood 1016.

Accordingly, as illustrated in FIG. 10, the determining of cochlear trauma likelihood 1016 may be performed (for action 522) by: 1) determining, based on accessed recipient attribute data, prediction model 1002 for the electrical potential at the estimated depth D₁ in an absence of cochlear trauma; 2) accessing historical data 1014 associated with a set of electrical potentials recorded during a plurality of previous insertion procedures, the plurality of previous insertion procedures including a first subset of insertion procedures determined to have resulted in cochlear trauma and a second subset of insertion procedures determined to have resulted in no cochlear trauma; 3) assessing the electrical potential (i.e., the measured potential 1004 recorded at depth D₁ to be response level 1008) in relation to the prediction model 1002 for the estimated depth D₁ (at response level 1006); and 4) determining likelihood 1016 based on a comparison, using a machine learning algorithm, of the assessment of the electrical potential in relation to the prediction model and one or more analogous assessments of historical data 1014.

This assessment of the electrical potential in relation to the prediction model and the historical data may be performed in any suitable way. For instance, a classification approach may be used in which machine learning engine 1012 is trained on a classification model that is based on historical data 1014 representing both the first subset of insertion procedures (determined to have resulted in cochlear trauma) and the second subset of insertion procedures (determined to have resulted in no cochlear trauma). In either example (or using another suitable approach), it will be understood that, for the case illustrated in FIG. 10 where the measured potential 1004 signal has veered so dramatically away from the prediction model 1002 at depth D₁, the cochlear trauma management system may determine that trauma is likely to be occurring because similar patterns can be observed in the historical cases that did end up resulting in trauma. By the same token, for other examples where measured potential 1004 and prediction model 1002 are not so different, the cochlear trauma management system may determine that trauma is unlikely since a pattern may be identified that more closely mimics historical patterns that are not associated with trauma.

As another example, an anomaly detection approach may be used in which machine learning engine 1012 is trained on an anomaly detection model that is based on accessed recipient attribute data and historical data 1014. Such a model may be applied to the accessed electrical potential (i.e., the measured potential 1004 recorded at depth D₁ to be response level 1008) so as to designate the electrical potential as traumatic if it does not follow the general pattern of the training data (i.e., if an anomaly is detected that would not be expected based on the historical data).

Machine learning engine 1012 may employ any suitable algorithm as may serve a particular implementation. For instance, for one-dimensional features, a machine learning algorithm such as Isolation Forests or Local Outlier Factor may be utilized to classify feedback signals as traumatic or atraumatic. As another example, deep learning methods (e.g., sequential or convolutional autoencoders) may be employed for multi-dimensional features.

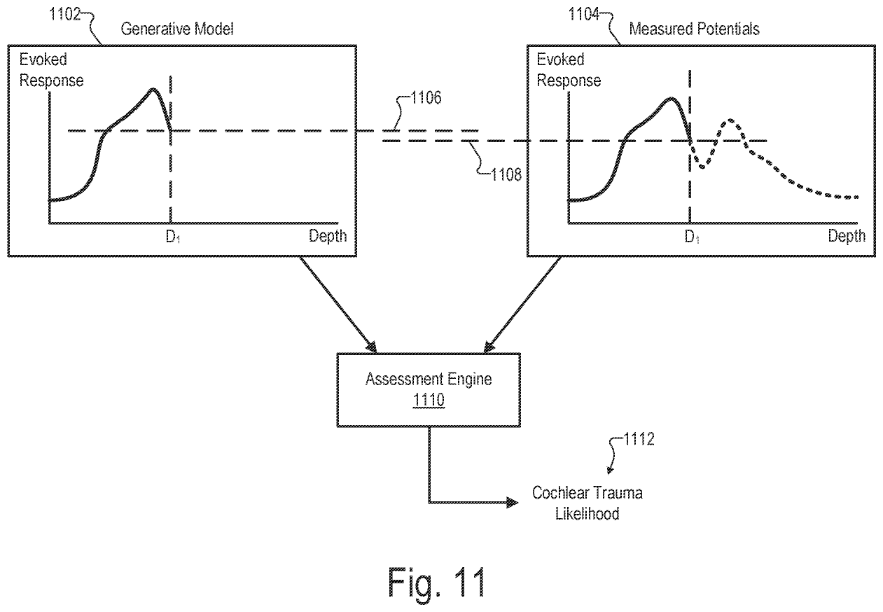

A third factor that may be taken into account while assessing the likelihood is the extent to which recorded electrical potentials align with a generative model of what trauma would be expected to look like in the diagnostic test results if trauma were indeed being inflicted (e.g., a model generated, for example, using a Bayesian inference procedure or another suitable algorithm). FIG. 11 illustrates how this “generative modeling factor” may be taken into account for an illustrative generative model that predicts what evoked responses will be measured if trauma is present.

More particularly, FIG. 11 shows a generative model 1102 that is customized for a particular cochlea of a particular recipient and, in contrast to prediction models 902 and 1002, represents a custom prediction of what evoked response data will look like, as a function of cochlear depth, if trauma is in fact being inflicted as the electrode lead is inserted. Generative model 1102 may be derived from continuous estimation of the parameters of a generative model of the effects of trauma on the feedback signal, using, for example, a Bayesian inference procedure. For instance, insertion trauma resulting in translocation of the electrode from one cochlear compartment to another may be modeled as a simultaneous 180° phase shift in the evoked responses at all frequencies. Similar to FIGS. 9 and 10, FIG. 11 also shows a parallel graph of measured potentials 1104 and a particular depth D₁ that is selected as an arbitrary depth for purposes of this example. When the electrode lead is at depth D₁ for this example, generative model 1102 may indicate that the electrical potential at this depth is predicted to be at a response level 1106 if trauma is occurring, while measured potentials 1104 indicates that the electrical potential actually recorded at this depth is at a response level 1108.

An assessment engine 1110 may take both of these levels into account (e.g., performing a hardware or software comparison to analyze the difference between the levels, etc.), but, rather than determining that cochlear trauma is likely if levels 1106 and 1108 are not similar enough, assessment engine 1110 may instead determine that cochlear trauma is likely if levels 1106 and 1108 are substantially similar (e.g., within a threshold, etc.). This is because, unlike the prediction models described above, generative model 1102 is configured to continuously model what the measured potentials will look like if trauma is present. This likelihood is output as cochlear trauma likelihood 1112.

Accordingly, as illustrated in FIG. 11, the determining of cochlear trauma likelihood 1112 is performed (for action 522) by: 1) determining, based on the recipient attribute data, a generative model 1102 indicative of a predicted effect of cochlear trauma on the electrical potential at the estimated depth D₁; and 2) determining likelihood 1112 based on a comparison of the electrical potential (measured potential 1104 recorded at response level 1108 at depth D₁) to the generative model (at response level 1106 at depth D₁). For example, in this case where the response level 1108 for measured potential 1104 is so similar to the response level 1106 that is anticipated if trauma is present, the cochlear trauma management system may determine that trauma is likely to be occurring, whereas if these level were more disparate, the system may determine that trauma is less likely.

In FIGS. 9-11, several different factors have been described and illustrated as potential factors that may be accounted for at action 522 to determine the likelihood that trauma is being inflicted. In various implementations, one or more of these factors may be accounted for to accurately, objectively, and reliably assess the likelihood in a manner that serves a particular implementation. In some implementations, a combination of factors may be analyzed and weighed in certain ways to come up with a numerical confidence value that represents the likelihood of trauma in a quantitative manner. Specifically, for example, the determination of the likelihood may be performed (for action 522) by: 1) assessing a first probability that the electrode lead is inflicting trauma on the cochlea with respect to a first trauma indication factor (e.g., one of the factors described above); 2) assessing a second probability that the electrode lead is inflicting trauma on the cochlea with respect to a second trauma indication factor different from the first trauma indication factor (e.g., a different one of the factors described above); and 3) generating, based on the first and second probabilities, a confidence value indicative of the likelihood that the electrode lead is inflicting trauma on the cochlea.

Returning to FIG. 5, after the likelihood of trauma is determined at action 522, action 524 may involve performing one or more feedback actions to utilize the likelihood determination in a productive way (e.g., to reduce trauma and improve the insertion procedure in real time). Specifically, in certain examples, action 524 may be performed based on the likelihood (determined at action 522) that the electrode lead is inflicting the trauma on the cochlea by providing, to a person participating in the insertion procedure (e.g., a surgeon or surgical team member, etc.) one or more of an audible indication of the likelihood, a visual indication of the likelihood, or a haptic indication of the likelihood. Additionally or alternatively, as another example, action 524 may be performed based on the likelihood that the electrode lead is inflicting the trauma on the cochlea by providing, to a robotic system participating in the insertion procedure, a signal indicative of the likelihood and configured to direct the robotic system to alter the insertion procedure (e.g., to slow or stop automatic advancement of the electrode lead, etc.).

To illustrate these feedback actions, FIG. 12 shows illustrative aspects of how feedback provided by a cochlear trauma management system may be used to ensure that an insertion procedure is effective and successful. Specifically, as shown by arrows 1202, respective likelihood indications may be provided by system 100 to either or both of a user 1204 (e.g., a person involved in performing the insertion procedure) and a robotic system 1206 (e.g., a system configured to perform automated actions in furtherance of the insertion procedure). As indicated by arrows 1208, either or both of user 1204 and robotic system 1206 may influence a particular insertion procedure 1210, which may then feedback information 1212 (e.g., evoked response data, etc.) to system 100. In this way, system 100 may improve insertion procedure 1210 by influencing user 1204 and/or robotic system 1206 with the data representative of how likely it is that cochlear trauma is being inflicted at any particular time during insertion procedure 1210. For example, upon receiving an indication that all is well (e.g., no visual, audible, or haptic alerts, etc.), user 1204 and robotic system 1206 may proceed with insertion procedure 1210 in an ordinary manner, but then, upon receiving an indication that trauma is likely to be occurring (e.g., as indicated by an alert or other indications described above), user 1204 and/or robotic system 1206 may make provisions in real time to proceed differently than they would without the indication from system 100. For instance, the electrode lead may be backed out somewhat and advanced in a different way or with a different approach, the lead advancement may be slowed or performed with increased caution, or the like.

Returning to FIG. 5, postoperative actions 526 and 528 may be performed to help the system learn from this insertion procedure and to allow future insertion procedures to be continually improved. Specifically, at action 526, a medical imaging assessment, a postoperative audiogram, or another suitable postoperative assessment may be made to determine whether the recipient did in fact experience any cochlear trauma as a result of the insertion procedure. For example, if a postoperative audiogram essentially lines up with the preoperative audiogram, it may be assumed that no trauma was inflicted, whereas if the postoperative audiogram reveals certain frequencies that are more difficult for the recipient to perceive postoperatively than preoperatively, it may be determined that some amount of trauma was inflicted. In either case, action 528 involves updating the historical data so that this insertion procedure and its outcome can be used in the future as another data point in determining, ever more accurately, whether cochlear trauma is being inflicted during the surgery of future recipients.

In certain embodiments, one or more of the processes described herein may be implemented at least in part as instructions embodied in a non-transitory computer-readable medium and executable by one or more computing devices. In general, a processor (e.g., a microprocessor) receives instructions, from a non-transitory computer-readable medium, (e.g., a memory, etc.), and executes those instructions, thereby performing one or more processes, including one or more of the processes described herein. Such instructions may be stored and/or transmitted using any of a variety of known computer-readable media.

A computer-readable medium (also referred to as a processor-readable medium) includes any non-transitory medium that participates in providing data (e.g., instructions) that may be read by a computer (e.g., by a processor of a computer). Such a medium may take many forms, including, but not limited to, non-volatile media, and/or volatile media. Non-volatile media may include, for example, optical or magnetic disks and other persistent memory. Volatile media may include, for example, dynamic random access memory (DRAM), which typically constitutes a main memory. Common forms of computer-readable media include, for example, a disk, hard disk, magnetic tape, any other magnetic medium, a compact disc read-only memory (CD-ROM), a digital video disc (DVD), any other optical medium, random access memory (RAM), programmable read-only memory (PROM), electrically erasable programmable read-only memory (EPROM), FLASH-EEPROM, any other memory chip or cartridge, or any other tangible medium from which a computer can read.

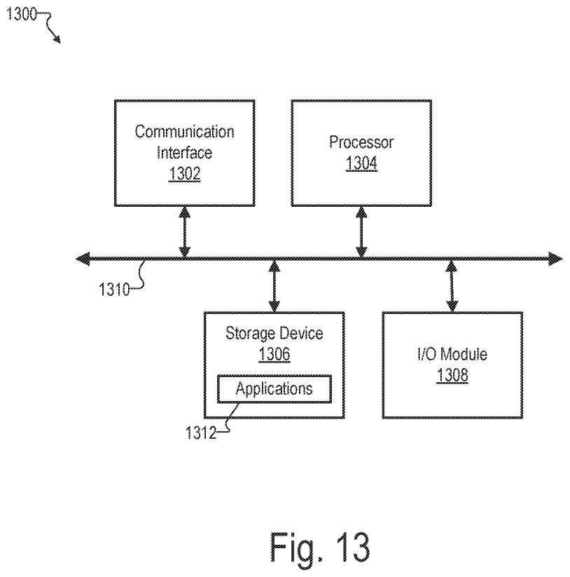

FIG. 13 shows an illustrative computing system 1300 that may implement cochlear trauma management systems and/or other computing systems and devices described herein. For example, computing system 1300 may include or implement (or partially implement) a cochlear trauma management system such as system 100, or any component included therein or system associated therewith. In some examples, computing system 1300 may be implemented or included within a cochlear implant system component (e.g., a sound processor, a headpiece, a cochlear implant, etc.) or a system associated with a cochlear implant system (e.g., computing device 312).

As shown in FIG. 13, computing system 1300 may include a communication interface 1302, a processor 1304, a storage device 1306, and an input/output (I/O) module 1308 communicatively connected via a communication infrastructure 1310. While an illustrative computing system 1300 is shown in FIG. 13, the components illustrated in FIG. 13 are not intended to be limiting. Additional or alternative components may be used in other embodiments. Components of computing system 1300 shown in FIG. 13 will now be described in additional detail.

Communication interface 1302 may be configured to communicate with one or more computing devices. Examples of communication interface 1302 include, without limitation, a wired network interface (such as a network interface card), a wireless network interface (such as a wireless network interface card), a modem, an audio/video connection, and any other suitable interface.

Processor 1304 generally represents any type or form of processing unit capable of processing data or interpreting, executing, and/or directing execution of one or more of the instructions, processes, and/or operations described herein. Processor 1304 may direct execution of operations in accordance with one or more applications 1312 or other computer-executable instructions such as may be stored in storage device 1306 or another computer-readable medium.

Storage device 1306 may include one or more data storage media, devices, or configurations and may employ any type, form, and combination of data storage media and/or device. For example, storage device 1306 may include, but is not limited to, a hard drive, network drive, flash drive, magnetic disc, optical disc, RAM, dynamic RAM, other non-volatile and/or volatile data storage units, or a combination or sub-combination thereof. Electronic data, including data described herein, may be temporarily and/or permanently stored in storage device 1306. For example, data representative of one or more executable applications 1312 configured to direct processor 1304 to perform any of the operations described herein may be stored within storage device 1306. In some examples, data may be arranged in one or more databases residing within storage device 1306.

I/O module 1308 may include one or more I/O modules configured to receive user input and provide user output. One or more I/O modules may be used to receive input for a single virtual experience. I/O module 1308 may include any hardware, firmware, software, or combination thereof supportive of input and output capabilities. For example, I/O module 1308 may include hardware and/or software for capturing user input, including, but not limited to, a keyboard or keypad, a touchscreen component (e.g., touchscreen display), a receiver (e.g., an RF or infrared receiver), motion sensors, and/or one or more input buttons.

I/O module 1308 may include one or more devices for presenting output to a user, including, but not limited to, a graphics engine, a display (e.g., a display screen), one or more output drivers (e.g., display drivers), one or more audio speakers, and one or more audio drivers. In certain embodiments, I/O module 1308 is configured to provide graphical data to a display for presentation to a user. The graphical data may be representative of one or more graphical user interfaces and/or any other graphical content as may serve a particular implementation.

In some examples, any of the facilities described herein may be implemented by or within one or more components of computing system 1300. For example, one or more applications 1312 residing within storage device 1306 may be configured to direct processor 1304 to perform one or more processes or functions associated with processor 104 of system 100. Likewise, memory 102 of system 100 may be implemented by or within storage device 1306.

In the preceding description, various exemplary embodiments have been described with reference to the accompanying drawings. It will, however, be evident that various modifications and changes may be made thereto, and additional embodiments may be implemented, without departing from the scope of the invention as set forth in the claims that follow. For example, certain features of one embodiment described herein may be combined with or substituted for features of another embodiment described herein. The description and drawings are accordingly to be regarded in an illustrative rather than a restrictive sense.

1. A system comprising:
a memory storing instructions; and
a processor communicatively coupled to the memory and configured to execute the instructions to:
determine, intraoperatively during an insertion procedure to introduce an electrode lead of a cochlear implant system into a cochlea of a recipient, an electrical potential evoked in response to acoustic stimulation applied to the recipient as part of a diagnostic test;
estimate a depth of the electrode lead within the cochlea, the estimated depth corresponding to the electrical potential resulting from the diagnostic test;
access recipient attribute data that includes etiological data representative of a mechanism by which the recipient experiences hearing loss and that represents a hearing attribute of the recipient that is correlated to the estimated depth of the electrode lead;
determine, intraoperatively during the insertion procedure and based on the electrical potential and the recipient attribute data, a likelihood that the electrode lead is inflicting trauma on the cochlea at the estimated depth; and
perform, intraoperatively during the insertion procedure, an action based on the likelihood that the electrode lead is inflicting the trauma on the cochlea.

2. The system of claim 1, wherein the diagnostic test during which the electrical potential is evoked is an electrocochleography (ECochG) test in which the electrical potential is recorded, within the cochlea, using an electrode of the electrode lead after the acoustic stimulation is applied.

3. The system of claim 1, wherein the estimating of the depth of the electrode lead within the cochlea includes:
detecting a first impedance of a first electrode included on the electrode lead;
detecting a second impedance of a second electrode included on the electrode lead, the second electrode adjacent to the first electrode so as to follow the first electrode into the cochlea as the electrode lead is introduced into the cochlea during the insertion procedure;
determining, based on the first and second impedances, that the first electrode has passed through a round window of the cochlea and that the second electrode has yet to pass through the round window; and
estimating the depth of the electrode lead based on the determining that the first electrode has passed through the round window and that the second electrode has yet to pass through the round window.

4. The system of claim 1, wherein the estimating of the depth of the electrode lead within the cochlea includes:
applying the acoustic stimulation as multi-tone stimulation including a first component at a first frequency associated with a first depth within the cochlea and a second component at a second frequency associated with a second depth within the cochlea;
determining, based on the electrical potential evoked in response to the acoustic stimulation, that an electrode used to record the electrical potential has passed the first depth associated with the first frequency and has yet to pass the second depth associated with the second frequency; and
estimating the depth of the electrode lead based on the determining that the electrode has passed the first depth and has yet to pass the second depth.

5. The system of claim 1, wherein the estimating of the depth of the electrode lead within the cochlea includes:
determining an amount of time that has elapsed during the insertion procedure since the electrode lead was at a known depth; and
estimating the depth of the electrode lead based on the known depth and the amount of time that has elapsed during the insertion procedure since the electrode lead was at the known depth.

6. The system of claim 1, wherein the recipient attribute data indicates a signal generator profile along the cochlea of the recipient.

7. The system of claim 6, wherein the signal generator profile indicated in the recipient attribute data is associated with a preoperative audiogram performed for the recipient prior to the insertion procedure.

8. (canceled)

9. The system of claim 1, wherein the determining of the likelihood that the electrode lead is inflicting trauma on the cochlea includes:
determining, based on the recipient attribute data, a prediction model for the electrical potential at the estimated depth in an absence of cochlear trauma;
determining, based on the prediction model, a threshold level that the electrical potential is predicted to exceed for the estimated depth in the absence of cochlear trauma; and
determining the likelihood based on whether the electrical potential exceeds the threshold level.

10. The system of claim 1, wherein the determining of the likelihood that the electrode lead is inflicting trauma on the cochlea includes:
determining, based on the recipient attribute data, a prediction model for the electrical potential at the estimated depth in an absence of cochlear trauma;
accessing historical data associated with a set of electrical potentials recorded during a plurality of previous insertion procedures, the plurality of previous insertion procedures including a first subset of insertion procedures determined to have resulted in cochlear trauma and a second subset of insertion procedures determined to have resulted in no cochlear trauma;
assessing the electrical potential in relation to the prediction model for the estimated depth; and
determining the likelihood based on a comparison, using a machine learning algorithm, of the assessment of the electrical potential in relation to the prediction model and one or more analogous assessments of the historical data.

11. The system of claim 1, wherein the determining of the likelihood that the electrode lead is inflicting trauma on the cochlea includes:
determining, based on the recipient attribute data, a generative model indicative of a predicted effect of cochlear trauma on the electrical potential at the estimated depth; and
determining the likelihood based on a comparison of the electrical potential to the generative model.

12. The system of claim 1, wherein the determining of the likelihood that the electrode lead is inflicting trauma on the cochlea includes:
assessing a first probability that the electrode lead is inflicting trauma on the cochlea with respect to a first trauma indication factor;
assessing a second probability that the electrode lead is inflicting trauma on the cochlea with respect to a second trauma indicator factor different from the first trauma indication factor; and
generating, based on the first and second probabilities, a confidence value indicative of the likelihood that the electrode lead is inflicting trauma on the cochlea.

13. The system of claim 1, wherein the action performed based on the likelihood that the electrode lead is inflicting the trauma on the cochlea includes providing, to a person participating in the insertion procedure, one or more of:
an audible indication of the likelihood;
a visual indication of the likelihood; or
a haptic indication of the likelihood.

14. The system of claim 1, wherein the action performed based on the likelihood that the electrode lead is inflicting the trauma on the cochlea includes providing, to a robotic system participating in the insertion procedure, a signal indicative of the likelihood and configured to direct the robotic system to alter the insertion procedure.

15. A system comprising:
a cochlear implant configured to be implanted within a recipient;
an electrode lead configured, upon being introduced into a cochlea of the recipient by way of an insertion procedure, to apply electrical stimulation to the recipient;
a loudspeaker configured to apply acoustic stimulation to the recipient; and
a processor configured to:
direct the cochlear implant to apply the electrical stimulation by way of the electrode lead and to direct the loudspeaker to apply the acoustic stimulation to the recipient as part of a diagnostic test;
determine, intraoperatively during the insertion procedure, an electrical potential evoked in response to the acoustic stimulation applied as part of the diagnostic test;
estimate a depth of the electrode lead within the cochlea, the estimated depth corresponding to the electrical potential resulting from the diagnostic test;
access recipient attribute data that includes etiological data representative of a mechanism by which the recipient experiences hearing loss and that represents a hearing attribute of the recipient that is correlated to the estimated depth of the electrode lead;
determine, intraoperatively during the insertion procedure and based on the electrical potential and the recipient attribute data, a likelihood that the electrode lead is inflicting trauma on the cochlea at the estimated depth; and
perform, intraoperatively during the insertion procedure, an action based on the likelihood that the electrode lead is inflicting the trauma on the cochlea.

16. The system of claim 15, wherein the diagnostic test during which the electrical potential is evoked is an electrocochleography (ECochG) test in which the electrical potential is recorded, within the cochlea, using an electrode of the electrode lead after the loudspeaker applies the acoustic stimulation.

17. The system of claim 15, wherein:
the recipient attribute data indicates a signal generator profile along the cochlea of the recipient, the signal generator profile associated with a preoperative audiogram performed for the recipient prior to the insertion procedure.

18. The system of claim 15, wherein the determining of the likelihood that the electrode lead is inflicting trauma on the cochlea includes:
determining, based on the recipient attribute data, a prediction model for the electrical potential at the estimated depth in an absence of cochlear trauma;
determining, based on the prediction model, a threshold level that the electrical potential is predicted to exceed for the estimated depth in the absence of cochlear trauma; and
determining the likelihood based on whether the electrical potential exceeds the threshold level.

19. The system of claim 15, wherein the determining of the likelihood that the electrode lead is inflicting trauma on the cochlea includes:
determining, based on the recipient attribute data, a prediction model for the electrical potential at the estimated depth in an absence of cochlear trauma;
accessing historical data associated with a set of electrical potentials recorded during a plurality of previous insertion procedures, the plurality of previous insertion procedures including a first subset of insertion procedures determined to have resulted in cochlear trauma and a second subset of insertion procedures determined to have resulted in no cochlear trauma;
assessing the electrical potential in relation to the prediction model for the estimated depth; and
determining the likelihood based on a comparison, using a machine learning algorithm, of the assessment of the electrical potential in relation to the prediction model and one or more analogous assessments of the historical data.

20. A method comprising:
determining, by a cochlear trauma management system and intraoperatively during an insertion procedure to introduce an electrode lead of a cochlear implant system into a cochlea of a recipient, an electrical potential evoked in response to acoustic stimulation applied to the recipient as part of a diagnostic test;
estimating, by the cochlear trauma management system, a depth of the electrode lead within the cochlea, the estimated depth corresponding to the electrical potential resulting from the diagnostic test;
accessing, by the cochlear trauma management system, recipient attribute data that includes etiological data representative of a mechanism by which the recipient experiences hearing loss and that represents a hearing attribute of the recipient that is correlated to the estimated depth of the electrode lead;
determining, by the cochlear trauma management system and intraoperatively during the insertion procedure and based on the electrical potential and the recipient attribute data, a likelihood that the electrode lead is inflicting trauma on the cochlea at the estimated depth; and
performing, by the cochlear trauma management system and intraoperatively during the insertion procedure, an action based on the likelihood that the electrode lead is inflicting the trauma on the cochlea.
